SEQUENCE LISTING (22 sequences)

SEQ ID NO: 1 - DNA, 21 bp, Artificial Sequence
  Synthetic oligonucleotide PAX3 forward
agctcggcggtgtttttatca

SEQ ID NO: 2 - DNA, 21 bp, Artificial Sequence
  Synthetic oligonucleotide PAX3 reverse
ctgcacaggatcttggagacg

SEQ ID NO: 3 - DNA, 22 bp, Artificial Sequence
  Synthetic oligonucleotide SNAI1 forward
actgcaacaaggaatacctcag

SEQ ID NO: 4 - DNA, 23 bp, Artificial Sequence
  Synthetic oligonucleotide SNAI1 reverse
gcactggtacttcttgacatctg

SEQ ID NO: 5 - DNA, 22 bp, Artificial Sequence
  Synthetic oligonucleotide SNAI2 forward
tgtgacaaggaatatgtgagcc

SEQ ID NO: 6 - DNA, 21 bp, Artificial Sequence
  Synthetic oligonucleotide SNAI2 reverse
tgagccctcagatttgacctg

SEQ ID NO: 7 - DNA, 21 bp, Artificial Sequence
  Synthetic oligonucleotide NGFR forward
cctacggctactaccaggatg

SEQ ID NO: 8 - DNA, 19 bp, Artificial Sequence
  Synthetic oligonucleotide NGFR reverse
cacacggtgttctgcttgt

SEQ ID NO: 9 - DNA, 21 bp, Artificial Sequence
  Synthetic oligonucleotide B3GAT1 forward
ctccttcgagaacttgtcacc

SEQ ID NO: 10 - DNA, 20 bp, Artificial Sequence
  Synthetic oligonucleotide B3GAT1 reverse
gggtcagtgaagcccttctt

SEQ ID NO: 11 - DNA, 21 bp, Artificial Sequence
  Synthetic oligonucleotide TFAP2A
actttgggtacgtgtgcgaaa

SEQ ID NO: 12 - DNA, 23 bp, Artificial Sequence
  Synthetic oligonucleotide TFAP2A reverse
cggaatgttgtcggttgagaaat

SEQ ID NO: 13 - DNA, 20 bp, Artificial Sequence
  Synthetic oligonucleotide SOX10 forward
cctcacagatcgcctacacc

SEQ ID NO: 14 - DNA, 23 bp, Artificial Sequence
  Synthetic oligonucleotide SOX10 reverse
catataggagaaggccgagtaga

SEQ ID NO: 15 - DNA, 19 bp, Artificial Sequence
  Synthetic oligonucleotide LRP1 forward
gagctgaaccacgcctttg

SEQ ID NO: 16 - DNA, 23 bp, Artificial Sequence
  Synthetic oligonucleotide LRP1 reverse
ggtagacactgccactccgatac

SEQ ID NO: 17 - DNA, 21 bp, Artificial Sequence
  Synthetic oligonucleotide LDLR forward
ggaccaacgaatgcttggaca

SEQ ID NO: 18 - DNA, 19 bp, Artificial Sequence
  Synthetic oligonucleotide LDLR reverse
ctggcacttgtagccaccc

SEQ ID NO: 19 - DNA, 22 bp, Artificial Sequence
  Synthetic oligonucleotide MME forward
agaagaaacagcgatggactcc

SEQ ID NO: 20 - DNA, 23 bp, Artificial Sequence
  Synthetic oligonucleotide MME reverse
catagagtgcgatcattgtcaca

SEQ ID NO: 21 - DNA, 22 bp, Artificial Sequence
  Synthetic oligonucleotide PLAT forward
atggatgcaatgaagagagggc

SEQ ID NO: 22 - DNA, 19 bp, Artificial Sequence
  Synthetic oligonucleotide PLAT reverse
ctgggcgaaacgaagactg

(19) United States
(12) Patent Application Publication
(10) Pub. No.: US 2015/0218523 A1
(43) Pub. Date: Aug. 6, 2015

(54) MANUFACTURE OF VASCULAR SMOOTH MUSCLE CELLS AND THE USE

(71) Applicant: Agency for Science, Technology and Research, Singapore (SG)

(72) Inventor: Christine Cheung, Singapore (SG)

(21) Appl. No.: 14/614,889

(22) Filed: Feb. 5, 2015

(30) Foreign Application Priority Data
Feb. 5, 2014 (SG) 2014008452

(51) Int. Cl. C12N 5/071

(52) U.S. Cl. CPC C12N 5/0691; C12N 2506/03; C12N 2501/115; C12N 2501/40; C12N 2501/999; C12N 2501/135; C12N 2501/15; C12N 2500/02

(57) ABSTRACT

A method for preparing brain-specific vascular smooth muscle cells comprising the step of: (a) contacting a population of stem cells with a composition comprising a bone morphogenetic protein (BMP) antagonist, a fibroblast growth factor (FGF) and an activin or nodal inhibitor to produce a population of neural crest cells.

### CROSS-REFERENCE TO RELATED APPLICATIONS

This application claims the benefit of priority of Singapore Patent Application No. 2014008452, filed Feb. 5, 2014, the contents of it being hereby incorporated by reference in its entirety for all purposes.

### FIELD OF THE INVENTION

The present invention generally relates to stem cell biology and drug screening. In particular, the invention covers the derivation of brains-specific vascular smooth muscle cells from stem cells, and the development of assays under hypoxic conditions for drug screening.

### BACKGROUND OF THE INVENTION

Amyloid plaques and neurofibrillary tangles are the primary hallmarks of Alzheimer's disease. There is an increasing recognition that the vascular system could play a causative role to some of these pathologies in the brain. The accumulation and deposition of amyloid-beta (Aβ) on the walls of blood vessels in the brain lead to cerebral amyloid angiopathy (CAA), which is implicated in patients with Alzheimer's disease and Down's syndrome. In normal physiology, cellular uptake and subsequent proteosomal degradation of Aβ are the principle means of metabolizing Aβ, with little accumulation in the central nervous system. Cerebrovascular smooth muscle cells (SMCs) and astrocytes are known to be able to remove Aβ locally via the low density lipoprotein receptor related protein 1 (LRP1)-mediated endocytic pathway. Brains of patients with severe CAA are often exposed to chronic hypoxia due to cerebral blood dysregulation and micro-haemorrhages. Hypoxia has been found to be associated with lowered LRP1 expression in cerebral SMCs, while LRP1 downregulation in vascular cells may also lead to dysfunctional local Aβ processing. It is therefore important to better understand these biological processes and to identify therapeutic interventions to target these neurovascular complexities.

Although animal models have been useful for the studies of diseases, judicious interpretation is required when extrapolating results from animal studies to human conditions due to inter-species differences. The traditional use of immortalized target-expressing cell lines and human primary cell lines for drug screening and biological studies suffers from limitations such as the lack of biological interactome in the former, and batch-to-batch variation in the latter. Furthermore, procurement of human brain vascular tissues for research is difficult and scarce.

There is thus a need to provide for an in vitro system that simulates the pathophysiological characteristics of cerebral vasculatures for biological studies and drug screening. It is also an objective of the present invention to provide sufficient quantities of cells suitable for biological studies and drug screening.

### SUMMARY OF THE INVENTION

In a first aspect, there is provided a method for preparing brain-specific vascular smooth muscle cells comprising the step of: (a) contacting a population of stem cells with a composition comprising a bone morphogenetic protein (BMP) antagonist, a fibroblast growth factor (FGF) and an activin or nodal inhibitor to produce a population of neural crest cells.

In a second aspect, there is provided a method for inducing a disease phenotype associated with abnormal amyloid-beta (Aβ) protein uptake and clearance comprising the step of exposing the brain-specific vascular SMCs prepared according to the method as defined herein to hypoxic condition for a length of time sufficient to induce said disease phenotype.

In a third aspect, there is provided a method for inducing a disease phenotype associated with abnormal amyloid-beta (Aβ) protein uptake and clearance comprising the step of exposing the brain-specific vascular SMCs prepared according to the method of any one of claims 1 to 16 to hypoxic condition for a length of time sufficient to induce said disease phenotype.

In a fourth aspect, there is provided a method for simulating or modeling a disorder associated with abnormal amyloid-beta (Aβ) protein uptake and clearance, comprising the method as defined herein.

In a fifth aspect, there is provided a method for screening a compound for ability to treat a condition associated with abnormal Aβ protein uptake and clearance, comprising the steps of:

(A) exposing the brain-specific vascular SMCs prepared according to the method as defined herein to the hypoxic condition specified as defined herein to induce a disease phenotype associated with abnormal Aβ protein uptake and clearance,

(B) contacting the exposed brain-specific vascular SMCs with said compound,

(C) further exposing the brain-specific vascular SMCs that have been contacted with said compound to Aβ protein for a time sufficient to enable Aβ uptake and clearance, and

(D) measuring the level of Aβ uptake and clearance in said brain-specific vascular SMCs and comparing the level of Aβ uptake and clearance in said brain-specific vascular SMCs with the level of Aβ uptake and clearance in brain-specific vascular SMCs that have not been contacted with said compound,

wherein an increased level of Aβ uptake and clearance in said brain-specific vascular SMCs that have been contacted with said compound compared to brain-specific vascular SMCs that have not been contacted with said compound indicates that said compound is useful for treating said condition,

and wherein a decreased or unchanged level of Aβ uptake and clearance in said brain-specific vascular SMCs that have been contacted with said compound compared to brain-specific vascular SMCs that have not been contacted with said compound indicates that said compound is not useful for treating said condition.

In a sixth aspect, there is provided a method for treating a patient in need of vascularized tissue replacement therapy, comprising administering to said patient the brain-specific vascular smooth muscle cells prepared according to the method as defined herein.

In a seventh aspect, there is provided a method of analyzing blood vessel development using the brain-specific vascular smooth muscle cells prepared according to the method as defined herein.

In an eight aspect, there is provided a method of determining the suitability of a compound for treating a patient having a condition associated with abnormal Aβ uptake and clearance, the method comprising preparing brain-specific vascular smooth muscle cells according to the method as defined herein using a population of stem cells derived from said patient in step (a), subjecting said brain-specific vascular smooth muscle cells to the method as defined herein to induce a disease phenotype associated with abnormal Aβ uptake and clearance, and determining the suitability of said compound for treating said patient using the method as defined herein.

In a ninth aspect, there is provided a composition comprising a BMP antagonist which is noggin present in a concentration of about 200 ng/ml, a FGF which is bFGF present in a concentration of about 12 ng/ml, an an activin or nodal inhibitor which is SB431542 present in a concentration of about 10 μM.

### BRIEF DESCRIPTION OF THE DRAWINGS

The invention will be better understood with reference to the detailed description when considered in conjunction with the non-limiting examples and the accompanying drawings, in which:

FIGS. 1A-1D: Differentiation potential of neural crest NGFR+/B3GAT1+ cells

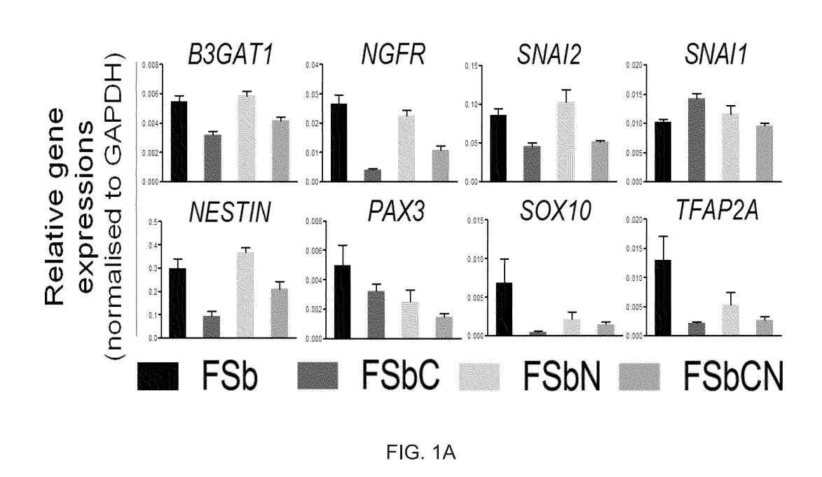

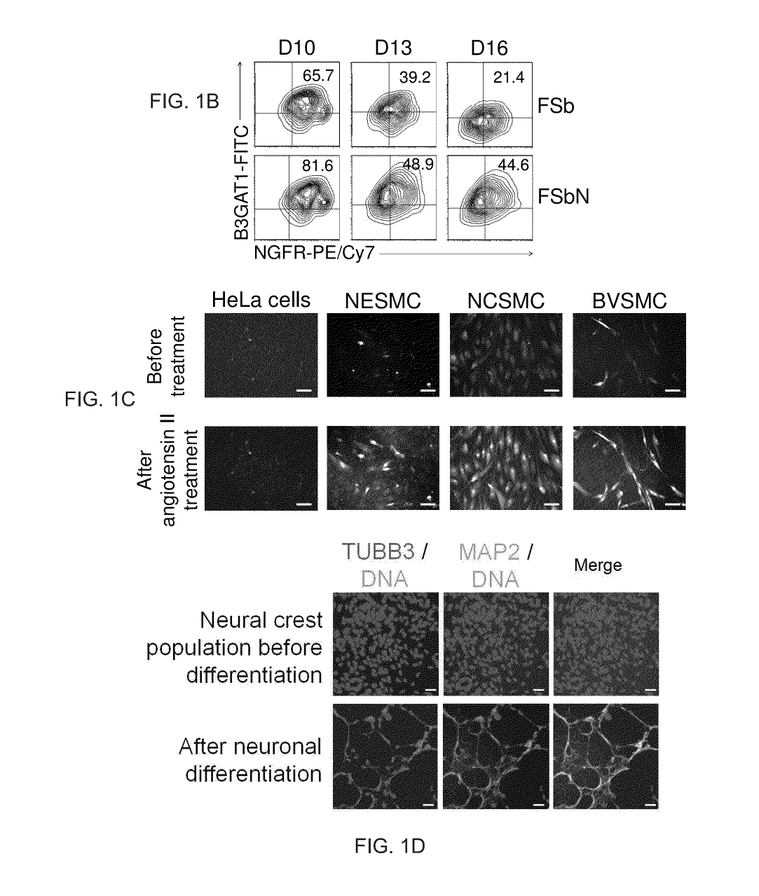

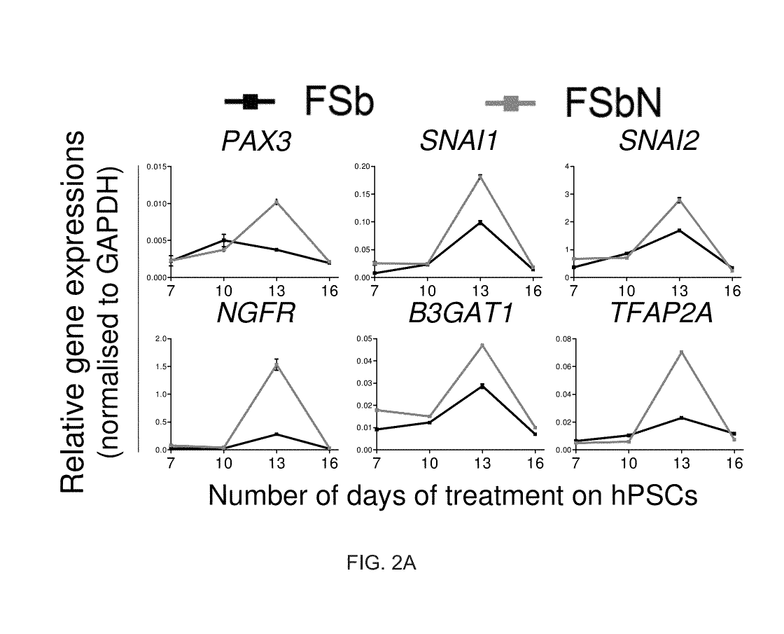

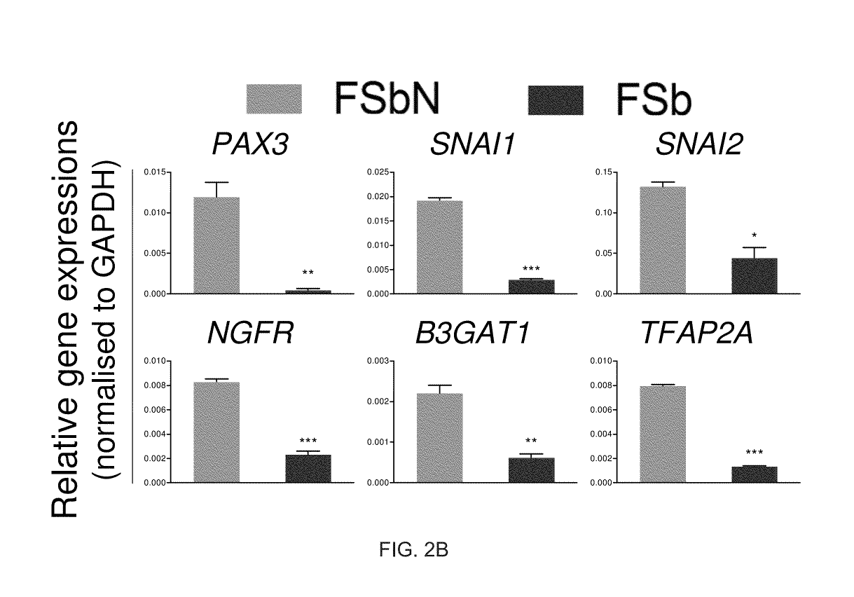

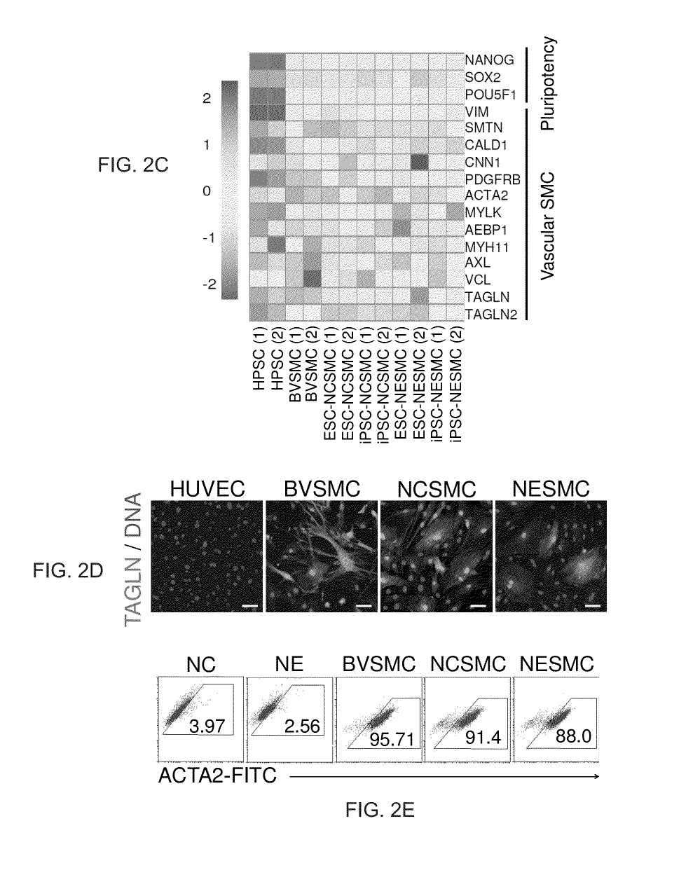

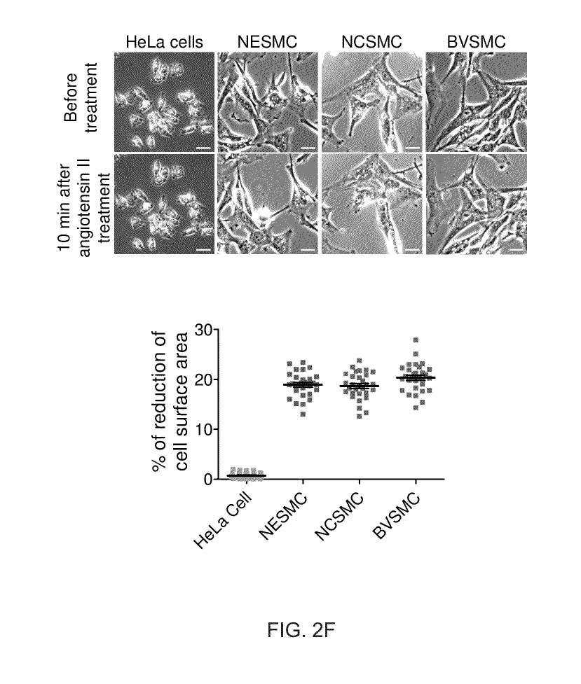

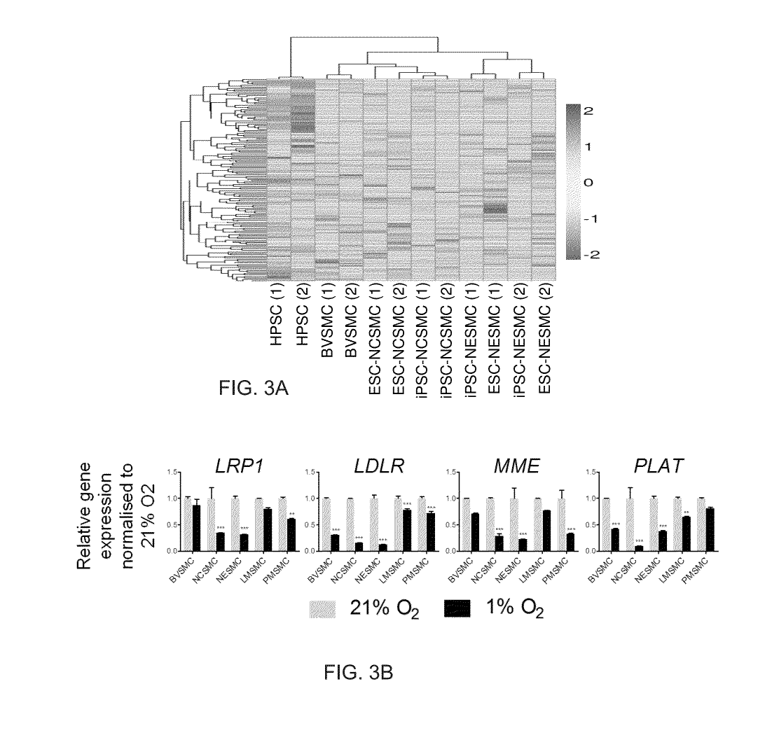

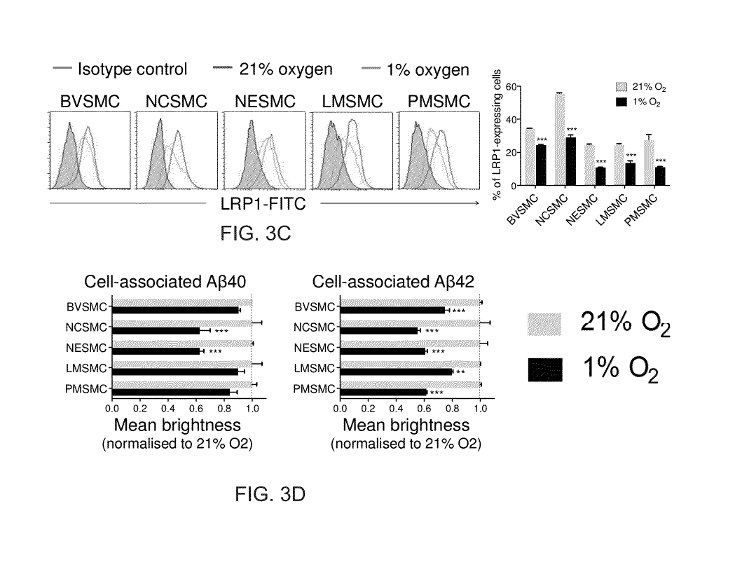

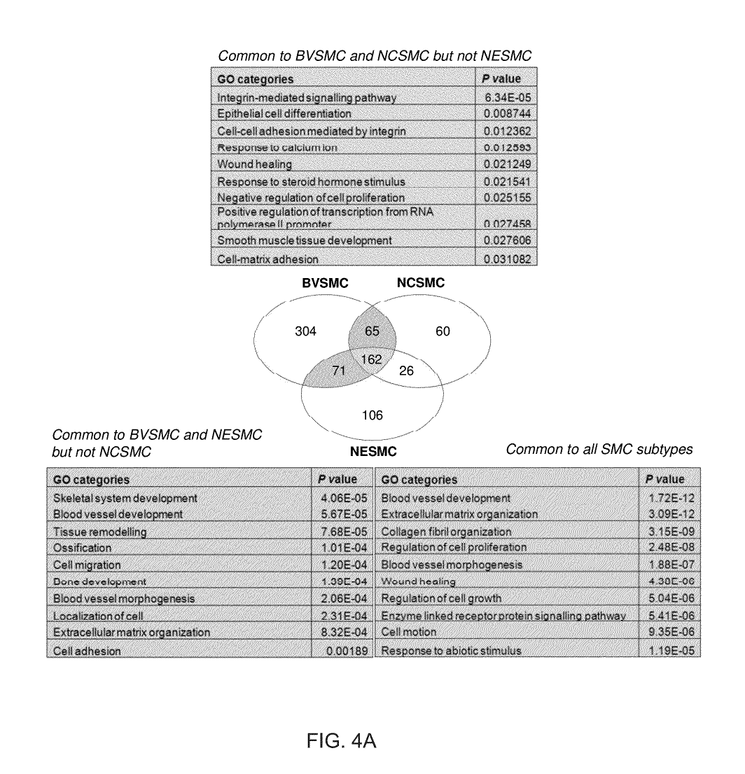

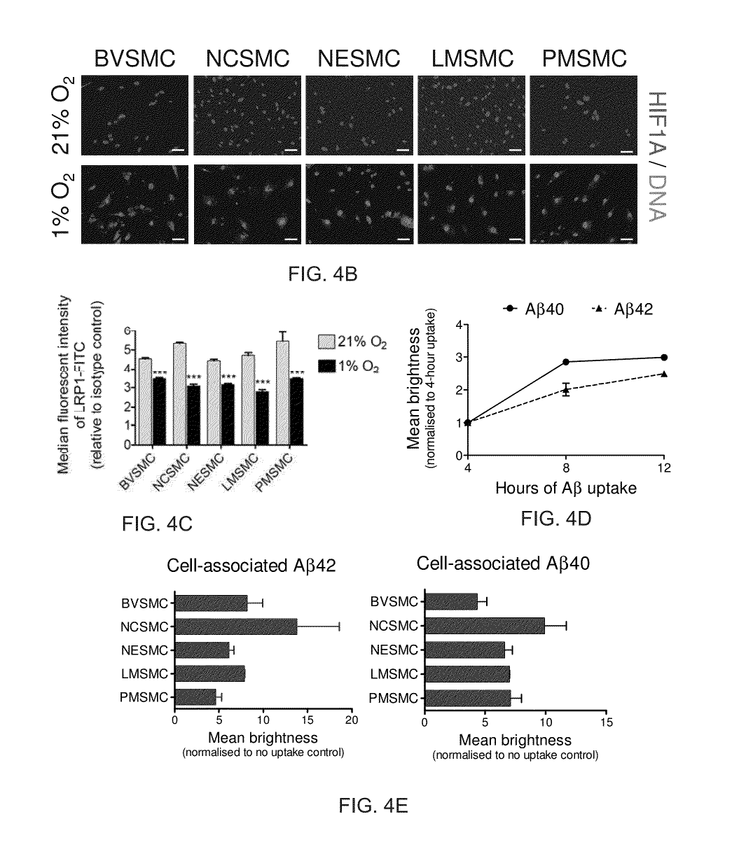

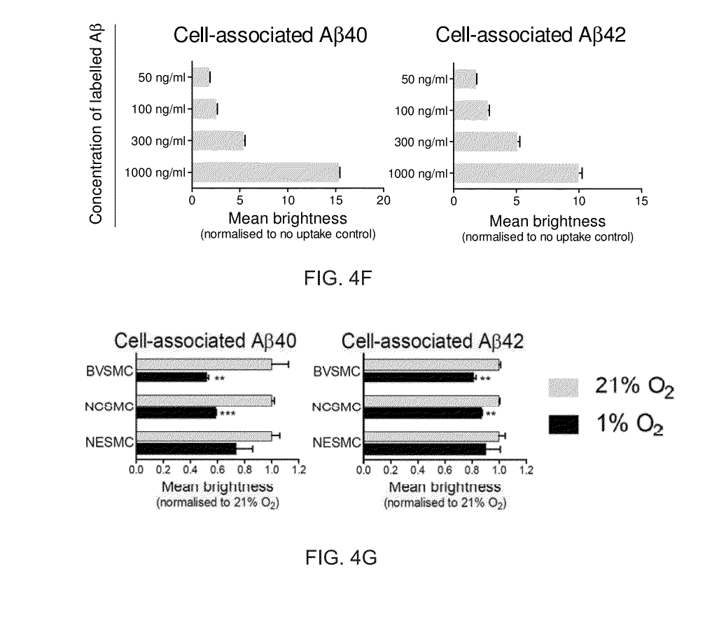

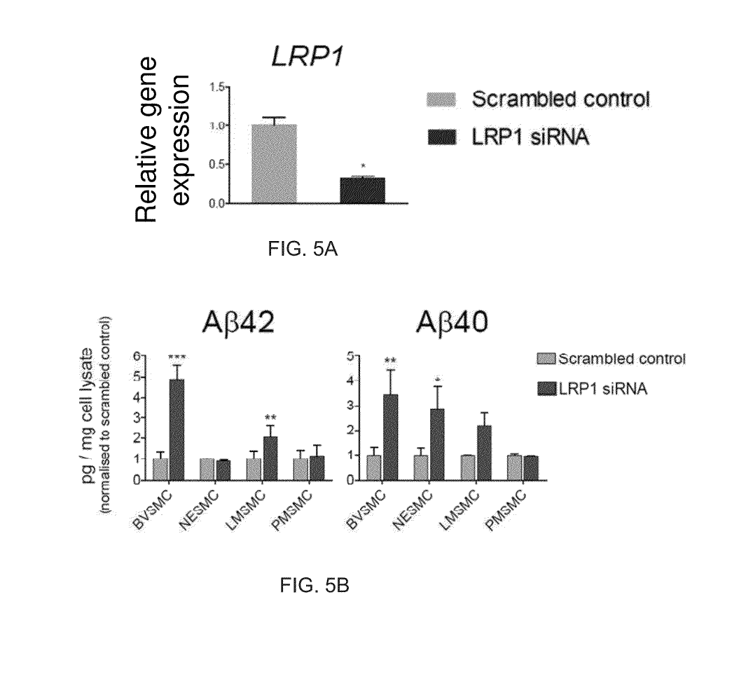

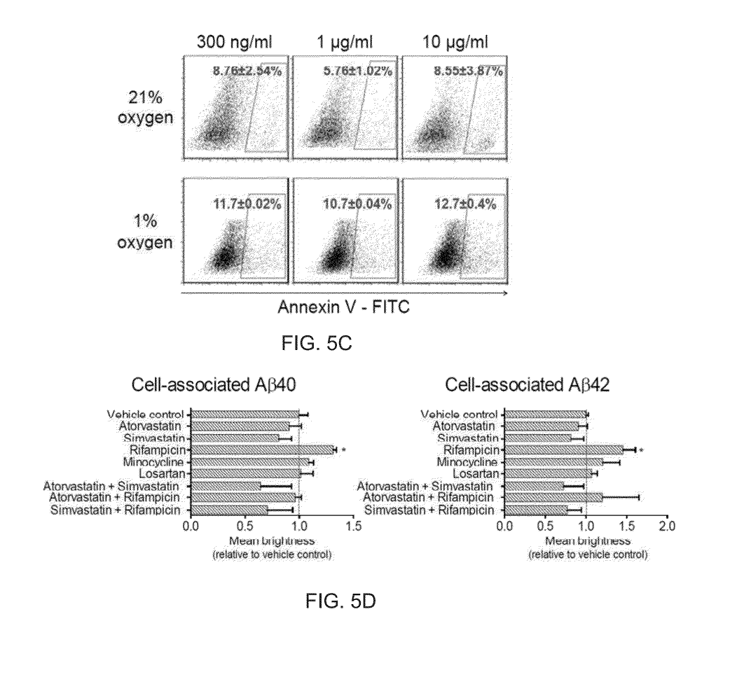

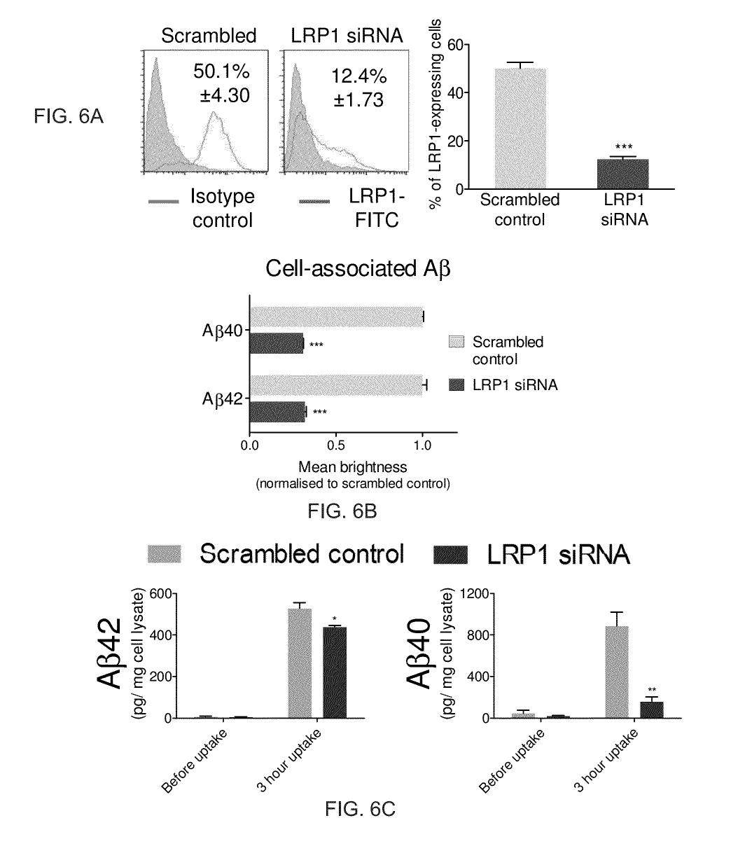

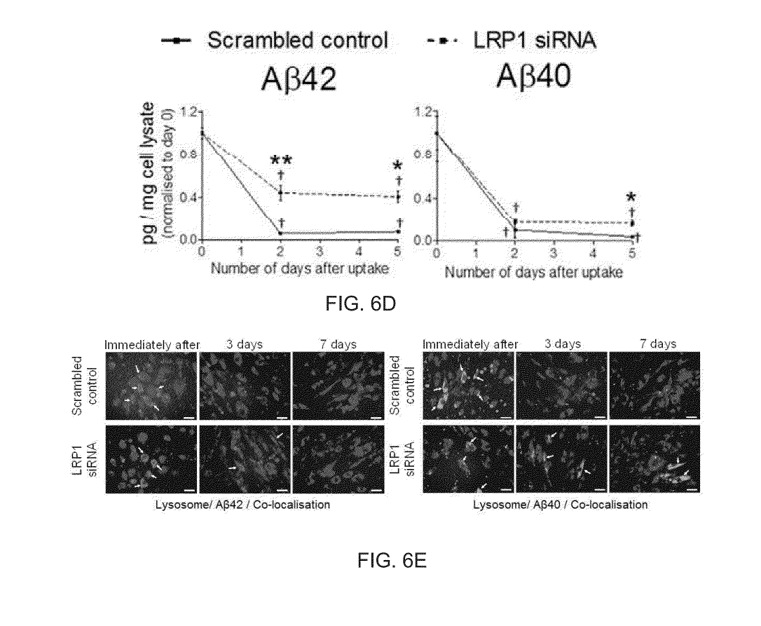

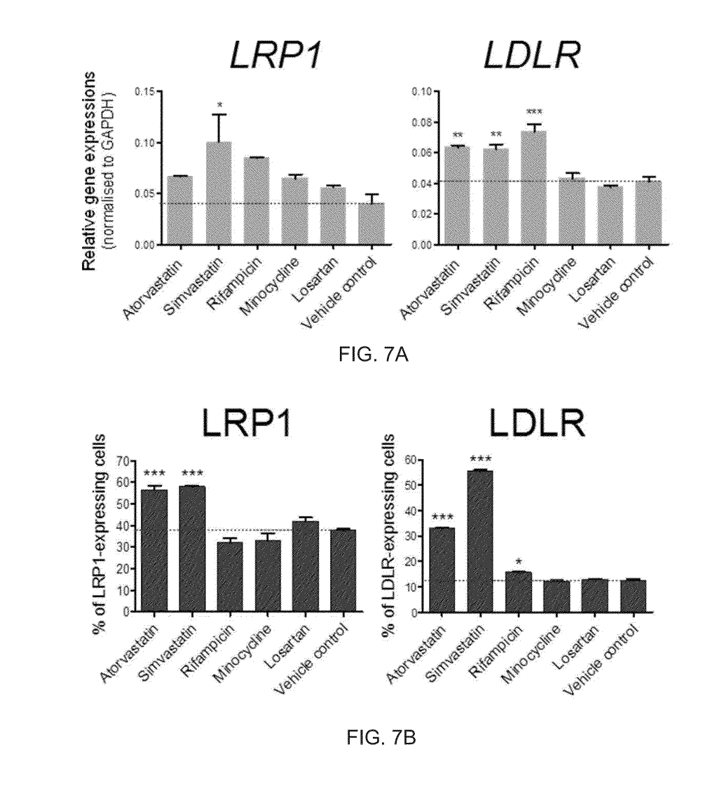

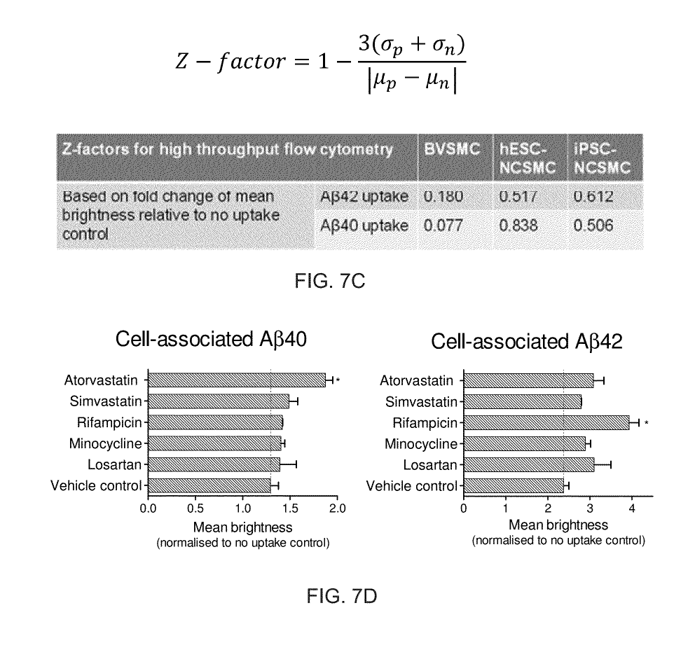

(FIG. 1A) qRT-PCR of neural crest markers in hPSCs (hESC H9) after 7 days of treatments in FSb (bFGF+SB431542), FSbC (bFGF+SB431542+CHIR99021), FSbN (bFGF+SB431542+noggin) and FSbCN (bFGF+SB431542+CHIR99021+noggin). Data represent means±s.e.m. (n=3).

(FIG. 1B) Flow cytometric analysis of neural crest surface proteins under FSb and FSbN treatments over time. Numbers in the top right hand quadrants represent percentages of doubly positive NGFR+/B3GAT1+ cells.

(FIG. 1C) Functional characterisation of neural crest-derived SMCs (NCSMC) compared to neuroectoderm-SMC (NESMC) and positive control, human brain vascular SMC (BVSMC). HeLa cells were used as a negative control. Fluo-4 loaded cells were monitored by time lapse microscopy over 10 min of treatment by angiotensin II, an inducer of contraction. All SMC subtypes demonstrated calcium signalling during contraction as indicated by fluo-4 fluorescence.

(FIG. 1D) Immunostaining for neuronal proteins confirmed neuronal differentiation potential of the isolated NGFR+/B3GAT1+ cells.

Scale bars represent 100 μM.

FIGS. 2A-2F: Characterizations of Neural Crest-Derived Vascular SMC

(FIG. 2A) Quantitative RT-PCR of neural crest markers during FSb (bFGF+SB431542) and FSbN (bFGF+SB431542+noggin) treatments on hPSCs. FSbN was more effective than FSb at inducing neural crest gene expressions between day 10 and day 16.

(FIG. 2B) FACS-sorted NGFR⁺/B3GAT1⁺ cells from FSb- and FSbN-treated populations were profiled for neural crest genes. Data represent means±SEM (n=3). Statistical differences compared to FSbN samples were calculated with Student's t test (*p<0.05, **p<0.01, **p<0.001).

(FIG. 2C) Microarray gene expression heatmap of SMC markers in hPSCs versus positive control, human brain vascular SMCs (BVSMCs), neural crest SMCs (NESMCs), and neuroectoderm SMCs (NESMCs) obtained from hESC (H9) and iPSCs (BJ-iPSCs) after differentiation. Orange (upregulation) and blue (downregulation) depict differential gene expression from the mean across all samples.

(FIG. 2D) NESMCs, NESMCs, and positive control, BVSMCs, were immunostained positively for SMC contractile protein, TAGLN. Human umbilical vein endothelial cells (HUVECs) were used as a negative control.

(FIG. 2E) Flow cytometric analysis of SMC protein, ACTA2, in NC and NE before SMC differentiation, as well as in NESMCs and NESMCs after differentiation. Numbers on the plots indicate percentage of ACTA2⁺ cells. A substantial proportion of cells in each SMC subtype was positive for ACTA2 (88%-95%).

(FIG. 2F) All SMC subtypes displayed contractile ability in response to angiotensin II. HeLa cells were used as a negative control. There was 18%-21% reduction of cell surface area in contracting SMCs. Data represent means±SEM (n=30). Scale bar, 50 mm. See also FIG. 1.

FIGS. 3A-3D: Chronic Hypoxia Compromises Ab Uptake Differentially in SMC Subtypes

(FIG. 3A) Clustering of genes from the gene ontology category of brain development revealed that the molecular signatures of NESMCs associated more closely with BVSMCs than NESMCs.

(FIG. 3B) A comparison of the effect of 21% versus 1% oxygen on the gene expressions of lipoprotein receptors and Ab-degrading enzymes in different SMC subtypes.

(FIG. 3C) Flow cytometric analysis of the percentage of LRP1+ cells in SMC subtypes. One percent oxygen significantly decreased LRP1 expression in all SMC subtypes. The black bars on the histogram plots demarcate the gating for quantifying the percentages of LRP1-expressing cells.

(FIG. 3D) Uptake of fluorescently labeled Aβ40 and Aβ42 peptides by SMC subtypes after 3 hr was measured by flow cytometry. Cell-associated Ab was calculated as mean brightness of the cells with Ab uptake as a ratio to their respective no-uptake control cells at 21% and 1% oxygen.

Data represent means±SEM (n=3). Statistical differences compared to 21% oxygen samples were calculated with ANOVA (*p<0.05, **p<0.01, ***p<0.001). Statistical differences compared to day 0 were calculated with ANOVA (yp<0.001). See also FIG. 4.

FIGS. 4A-4G: SMC subtypes demonstrated differential Aβ uptake ability

(FIG. 4A) Based on global gene expression profiles, the Venn diagram represent subsets of genes that were differentially upregulated in the SMC subtypes compared to hPSC (false-discovery rate, 0.05). The commonly upregulated genes in coloured subsets were analysed using the functional annotation clustering from DAVID bioinformatics resources. Top 10 highly enriched categories in each subset were indicated in the correspondingly coloured tables.

(FIG. 4B) Hypoxic treatment on the SMC subtypes. All SMC subtypes showed positive HIF1A staining after more than 2 weeks of culture at 1% oxygen. Scale bar represents 100 μm.

(FIG. 4C) A measure of median fluorescent intensity of LRP protein expressions in SMC subtypes under 21% versus 1% oxygen.

(FIG. 4D) A time course of uptake of fluorescently labelled Aβ40 and Aβ42 peptides by BVSMC. Aβ uptake was measured by flow cytometry as the mean brightness of cells relative to the 1-hour time point.

(FIG. 4E) Uptake of labelled Aβ peptides by SMC subtypes after 3 hours was measured by flow cytometry. Cell-associated Aβ was calculated as the mean brightness of cells with Aβ uptake as a ratio to their respective no uptake control cells.

(FIG. 4F) A range of concentrations of labelled Aβ peptides was tested for 3-hour uptake in NCSMC.

(FIG. 4G) Effect of hypoxia on the uptake of near-physiological concentration of Aβ peptides (300 ng/ml) in SMC subtypes. Cell-associated Aβ was measured by flow cytometry after 3 hours.

Data represent means±s.e.m. (n=3). Statistical differences compared to 21% oxygen samples were calculated with ANOVA (**P<0.01; ***P<0.001).

FIGS. 5A-5D: LRP1-mediated Aβ metabolism in hPSC-derived NCSMC

(FIG. 5A) LRP1 gene silencing in NCSMC which were grown routinely at 21% oxygen. qRT-PCR of lipoprotein receptor, LRP1, in NCSMC after 48 hours of LRP1 siRNA and scrambled siRNA treatments.

(FIG. 5B) Intracellular Aβ in SMC subtypes was monitored by ELISA 2 days after a 3-hour uptake of human recombinant Aβ peptides.

(FIG. 5C) Flow cytometric analysis of annexin V staining on Aβ40-treated NCSMC (4-day treatment) demonstrated that hypoxic condition exacerbated apoptosis of NCSMC. Increasing concentrations of Aβ40 only led to slight increase of apoptotic NCSMC at 1% oxygen.

(FIG. 5D) Reference drugs were treated on human iPSC (KYOUDXR0109B)-derived NCSMC for 48 hours prior to uptake of fluorescently labelled Aβ peptides for an hour in a 96-well plate format. Cell-associated Aβ was evaluated by high-throughput flow cytometry.

Data represent means±s.e.m. (n=3). Statistical differences compared to controls were calculated with Student's t-test (*P<0.05) or ANOVA (*P<0.05, **P<0.01; ***P<0.001).

FIGS. 6A-6E: LRP1 Mediates Ab Clearance in NCSMCs

(FIG. 6A) Flow cytometric analysis confirmed more than 3-fold LRP1 protein knockdown in LRP1 siRNA-treated NCSMCs compared to scrambled control.

(FIG. 6B) LRP1 silencing in NCSMCs resulted in significantly reduced uptake of fluorescently labeled Ab peptides in 3 hr as measured by flow cytometry.

FIG. 6C) ELISA validated the decreased internalization of exogenous recombinant Ab peptides in LRP1-suppressed NCSMCs after 3 hr uptake.

(FIG. 6D) Intracellular Ab was monitored over time by ELISA on NCSMC cell lysates after 3 hr uptake of recombinant Ab peptides. Ab degradation was evident from the drop in Ab levels by day 2, but NCSMCs with LRP1 knockdown were not as effective as scrambled controls in the elimination of internalized Ab peptides.

(FIG. 6E) NCSMCs were imaged at days 0, 3, and 7 after a 3 hr uptake of fluorescently labeled Ab40 (green) and Ab42 (red) peptides. White arrows indicate incorporation of labeled Aβ in lysosomes (blue). Scale bar, 100 mm. Data represent means±SEM (n=3). Statistical differences compared to scrambled controls were calculated with Student's t test for two groups or by ANOVA for three or more groups (*p<0.05, **p<0.01, **p<0.001). See also FIG. 5.

FIGS. 7A-7D: Testing of Vascular Protective Compounds in a High-Throughput Aβ Uptake Assay

(FIG. 7A) Reference drugs were treated on NCSMCs at 1% oxygen for 48 hr to investigate whether the compounds could rescue the compromised gene expressions of lipoprotein receptors by chronic hypoxia.

(FIG. 7B) Flow cytometric analysis of LRP1 and LDLR protein expression after 48 hr of compound treatment on NCSMCs at 1% oxygen.

(FIG. 7C) Z factor is a statistical measurement of the feasibility of a cell-based phenotypic assay for high-throughput screening. Average measurements on “fold change of mean brightness relative to no-uptake control” and SDs were obtained from positive (grown at 21% oxygen) and negative (grown at 1% oxygen) controls. The means and SDs of both positive (p) and negative (n) controls are indicated as (μₚ, σp) and (μₙ, σᵣ), respectively. A value of more than 0.5 indicates a robust Aβ uptake assay, whereas a value of between 0 and 0.5 is a marginal assay.

(FIG. 7D) Reference drugs were treated on NCSMCs for 48 hr prior to uptake of fluorescently labeled Ab peptides for an hour in a 96-well plate format. Cell-associated Aβ was evaluated by high-throughput flow cytometry.

Data represent means±SEM (n=3). Statistical differences compared to vehicle controls were calculated with Student's t test (*p<0.05, **p<0.01, **p<0.001). See also FIG. 5.

### DETAILED DESCRIPTION OF THE PRESENT INVENTION

In a first aspect, there is provided a method for preparing brain-specific vascular smooth muscle cells comprising the step of: (a) contacting a population of stem cells with a composition comprising a bone morphogenetic protein (BMP) antagonist, a fibroblast growth factor (FGF) and an activin or nodal inhibitor to produce a population of neural crest cells. Alternatively, the composition may comprise i) a bone morphogenetic protein (BMP) antagonist and a fibroblast growth factor (FGF); (ii) a bone morphogenetic protein (BMP) antagonist and an activin or nodal inhibitor; or (iii) a fibroblast growth factor (FGF) and an activin or nodal inhibitor.

The population of neural crest cells may be a heterogeneous or homogeneous population. The population of neural crest cells may comprise a sub-population of CD57+ cells or CD271+ neural crest progenitor cells. The population of neural crest cells may also comprise a sub-population of CD57+ and CD271+ neural crest progenitor cells. Progenitor cells may refer to cells with the ability to differentiate into specific types of cells. Neural crest progenitor cells may refer to a neural crest cells that have the potential to differentiate into other cell types, such as brain-specific vascular smooth muscle cells.

The term “brain-specific smooth muscle cells” may refer to smooth muscle cells that are closely related or the same as smooth muscle cells that are found in the brain. These “brain-specific smooth muscle cells” may have characteristics such as being contractile or may express smooth muscle cell genes and proteins that are similar or the same as smooth muscle cells that are found in the brain. The “brain-specific smooth muscle cells” may be induced to have a diseased phenotype under conditions such as hypoxia.

The method further comprises the step of (b) isolating a population of CD57+ and/or CD271+ neural crest progenitor cells from the population of neural crest cells produced in step (a). The population of CD57+ and/or CD271+ neural crest progenitor cells may be isolated using Fluorescence Assisted Cell Sorting (FACS) or magnetic assisted cell sorting (MACs).

The method may further comprises the step of: (c) contacting the population of neural crest progenitor cells with a composition comprising platelet-derived growth factor (PDGF) and transforming growth factor (TGF) to produce said brain-specific vascular smooth muscle cells.

The population of stem cells in step (a) may be human pluripotent stem cells. The human pluripotent stem cells may include, but is not limited to human embryonic stem cells (hESCs) or induced pluripotent stem cells (IPSCs). A stem cell may be a cell that has the potential to differentiate to a variety of different or specialized cell types. It also has the ability to divide to produce more stem cells. A pluripotent stem cell may refer to a stem cell that is able to differentiate to a cell of any of the three germ layers, i.e. endoderm, mesoderm and ectoderm. A totipotent stem cell, on the other hand, may refer to a stem cell that is able to differentiate to all different cell types, including a cell of any of the three germ layers (endoderm, mesoderm and ectoderm) as well as the cytotrophoblast layer or syncytiotrophoblast layer of the placenta. An induced pluripotent stem cell (IPSC) may refer to a pluripotent stem cell that is derived directly from an adult cell. The term “derive” may refer to obtaining from a particular source, such as from an adult cell. An “adult cell” may refer to a partially or fully differentiated cell. For example, an “adult cell” may be an adult stem cell such as a keratinocyte or fibroblast cell. An “adult cell” may also be, for example, a fully differentiated epithelial cell. An induced pluripotent stem cell may be derived from an adult cell by reprogramming of the adult cell by expression of exogenous factors, such as transcription factors. For example, a human induced pluripotent stem cell may be reprogrammed from a dermal fibroblast cell by the expression of OCT4, SOX2, KLF4 and MYC by retroviral transduction.

The human pluripotent stem cells, in particular the human embryonic stem cells, may be derived from stem cell lines and/or via stem cell preparation methods that do not involve destruction of human embryos. The human pluripotent stem cells may also be derived from a human embryo that is less than 14 days old. Alternatively, the population of stem cells may be of a non-human origin. For example, the population of stem cells may be primate or rodent pluripotent stem cells. The primate or rodent pluripotent stem cells may include, but is not limited to primate/rodent embryonic stem cells or induced pluripotent stem cells.

The contacting of the population of stem cells with the composition in step (a) may be carried out in a monolayer of said population of stem cells. Alternatively, the contacting of the population of stem cells with the composition in step (a) may be carried out in a suspension of said population of stem cells.

The composition in step (a) may not comprise a glycogen synthase kinase (GSK) inhibitor or a Wnt protein or a composition that is serum-free. In one example, such a glycogen synthase kinase (GSK) inhibitor is (2′Z, 3′E)-6-bromoindirubin-3′-oxime (BIO). In another example, such a Wnt protein is Wnt3a.

A bone morphogenetic protein (BMP) antagonist may refer to a molecule which inhibits or attenuates the biological activity of the BMP signaling pathway. Antagonists may include proteins such as antibodies, anticalins, nucleic acids, carbohydrates, small molecules, or any other compound or composition which modulates the activity of BMP either by directly interacting with BMP or by acting on components of the biological pathway in which BMP participates, such as a BMP receptor protein (e.g. BMP type I receptors ALK2 and/or ALK3) or downstream SMAD proteins).

An activin or nodal inhibitor may refer to a molecule which decreases the activity of the activin or nodal signaling pathway or decreases the protein levels of activin or nodal. Thus, a actin/nodal inhibitor can be a molecule which decreases the signaling activity of the activin or nodal proteins, e.g., by interfering with interaction of the activin/nodal with another molecule, such as an activin/nodal receptor (e.g. activin receptor-like kinase receptors such as ALK5 (TGFβ1 receptor), ALK4 and ALK7) It can also be a molecule which decreases expression of the gene encoding the activin or nodal proteins. An inhibitor can also be an antisense nucleic acid, a ribozyme, or an antibody.

A fibroblast growth factor (FGF) may refer to a member protein of the fibroblast growth factor (FGF) family or it may refer to a derivative or a polypeptide fragment of a member protein of the fibroblast growth factor (FGF) family.

The composition in step (a) may comprise:

(i) a BMP antagonist can include, but is not limited to noggin, an inhibitor of the transcriptional activity of the BMP type I receptors ALK2 and/or ALK3, chordin, or LDN193189; and/or

(ii) a FGF can include, but is not limited to a basic fibroblast growth factor (bFGF), FGF-17, FGF-5, FGF-16, FGF-6, FGF-20, FGF-12, FGF-4, FGF-10, FGF-21, FGF-8a, FGF-23, FGF-9, FGF-19, FGF-22, or FGF-3; and/or

(iii) an activin or nodal inhibitor can include but is not limited to SB431542, LY2157299, SB525334, SB505124 or LY2109761.

The composition in step (a) may comprise:

(i) a BMP antagonist present in a concentration that can include, but is not limited to about 100 ng/ml to about 500 mg/ml, about 100 ng/ml, about 200 ng/ml, about 300 ng/ml, about 400 ng/ml, or about 500 ng/ml; and/or

(ii) a FGF present in a concentration that can include, but is not limited to about 5 ng/ml to about 20 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml or about 20 ng/ml; and/or

(iii) an activin or nodal inhibitor present in a concentration that can include, but is not limited to about 5 μM to about 20 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, or about 20 μM.

The composition in step (a) may comprise:

(i) a BMP antagonist which is noggin present in a concentration of about 200 ng/ml; and/or

(ii) a FGF which is bFGF present in a concentration of about 12 ng/ml; and/or
(iii) an activin or nodal inhibitor which is SB431542 present in a concentration of about 10 μM.

The composition in step (a) may comprise:

(i) a BMP antagonist which is noggin present in a concentration of about 200 ng/ml; and/or

(ii) a FGF present in a concentration that can include, but is not limited to about 5 ng/ml to about 20 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml or about 20 ng/ml; and/or

(iii) an activin or nodal inhibitor present in a concentration that can include, but is not limited to about 5 μM to about 20 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, or about 20 μM.

The composition in step (a) may comprise:

(i) a BMP antagonist present in a concentration that can include, but is not limited to about 100 ng/ml to about 500 mg/ml, about 100 ng/ml, about 200 ng/ml, about 300 ng/ml, about 400 ng/ml, or about 500 ng/ml; and/or

(ii) a FGF present which is bFGF present in a concentration of about 12 ng/ml; and/or

(iii) an activin or nodal inhibitor present in a concentration that can include, but is not limited to about 5 μM to about 20 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, or about 20 μM.

The composition in step (a) may comprise:

(i) a BMP antagonist present in a concentration that can include, but is not limited to about 100 ng/ml to about 500 mg/ml, about 100 ng/ml, about 200 ng/ml, about 300 ng/ml, about 400 ng/ml, or about 500 ng/ml; and/or

(ii) a FGF present in a concentration that can include, but is not limited to about 5 ng/ml to about 20 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml or about 20 ng/ml; and/or

(iii) an activin or nodal inhibitor which is SB431542 present in a concentration of about 10 μM.

The contacting in step (a) may be for a duration that can include, but is not limited to about 8 days to about 18 days, about 10 days to about 16 days, about 8 days, about 9 days, about 10 days, about 11 days, about 12 days, about 13 days, about 14 days, about 15 days, about 16 days, about 17 days or about 18 days. In one embodiment, the duration is about 8 days to 18 days.

The composition in step (c) may comprise:

(i) a PDGF which is platelet-derived growth factor BB (PDGF-BB); and/or

(ii) a TGF which is transforming growth factor-beta 1 (TGF-β1).

The composition in step (c) may comprise:

(i) a PDGF present in a concentration that can include, but is not limited to about 5 ng/ml to about 20 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, or about 20 ng/ml; and/or

(ii) a TGF present in a concentration that can include, but is not limited to about 1 ng/ml to about 5 ng/ml, about 1 ng/ml, about 1.5 ng/ml, about 1.6 ng/ml, about 1.7 ng/ml, about 1.8 ng/ml, about 1.9 ng/ml, about 2 ng/ml, about 2.1 ng/ml, about 2.2 ng/ml, about 2.3 ng/ml, about 2.4 ng/ml, about 2.5 ng/ml, about 3 ng/ml, about 4 ng/ml or about 5 ng/ml. In one embodiment, the PDGF is present in a concentration of about 5 ng/ml to about 20 ng/ml and the TGF is present in a concentration of about 1 ng/ml to about 5 ng/ml.

In one embodiment, the PDGF is present in a concentration of about 10 ng/ml and the TGF is present in a concentration of about 2 ng/ml.

The composition in step (c) may comprise:

(i) a PDGF present in a concentration that can include, but is not limited to about 5 ng/ml to about 20 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, or about 20 ng/ml; and/or (ii) a TGF present in a concentration of about 2 ng/ml.

The composition in step (c) may comprise:

(i) a PDGF present in a concentration of about 10 ng/ml; and/or (ii) a TGF present in a concentration that can include, but is not limited to about 1 ng/ml to about 5 ng/ml, about 1 ng/ml, about 1.5 ng/ml, about 1.6 ng/ml, about 1.7 ng/ml, about 1.8 ng/ml, about 1.9 ng/ml, about 2 ng/ml, about 2.1 ng/ml, about 2.2 ng/ml, about 2.3 ng/ml, about 2.4 ng/ml, about 2.5 ng/ml, about 3 ng/ml, about 4 ng/ml or about 5 ng/ml.

The contacting in step (c) may be for a duration that can include, but is not limited to about 9 days to about 20 days, about 10 days to about 16 days, about 10 days to about 14 days, about 9 days, about 10 days, about 11 days, about 12 days, about 13 days, about 14 days, about 15 days, about 16 days, about 17 days, about days, about 19 days or about 20 days. In one embodiment, the duration is about 9 days to about 20 days. In one embodiment, the duration is about 12 days.

In one aspect, there is provided a method for inducing a disease phenotype associated with abnormal amyloid-beta (Aβ) protein uptake and clearance comprising the step of exposing the brain-specific vascular SMCs prepared according to the method as defined herein to hypoxic condition for a length of time sufficient to induce said disease phenotype. Abnormal amyloid-beta (Aβ) protein uptake and clearance may refer to lower than physiological rates of amyloid-beta (Aβ) protein uptake and clearance.

A hypoxic condition refers to a condition in which there is low level of oxygen as compared to normal physiological conditions (i.e. about 21% of oxygen). The hypoxic condition may comprise a condition that can include, but is not limited to about 5% of oxygen, less than about 4% of oxygen, less than about 3% of oxygen, less than about 2% of oxygen, or less than about 1% of oxygen. In one embodiment, the hypoxic condition comprises a condition of less than about 1% oxygen.

The length of time sufficient to induce said disease phenotype may include, but is not limited to at least about 48 hr, at least about 3 days, at least about 4 days, at least about 5 days, at least about 6 days, at least about 1 week, at least about 2 weeks, at least about 3 weeks, at least about 4 weeks, at least about 2 months, at least about 3 months, at least about 4 months, at least about 5 months, about 2 weeks or more, about 3 weeks or more, about 4 weeks or more, about 2 months or more, about 3 months or more, about 4 months or more, or about 5 months or more. In one embodiment, the length of time sufficient to induce said disease phenotype is at least about 2 weeks.

The Aβ protein may include, but is not limited to Aβ40 or Aβ42.

The disease phenotype may include, but is not limited to aging, neurological disorders, or cerebrovascular disorders. Neurological disorders may include, but are not limited to cerebral amyloid angioplasty, Alzheimer's disease, Down's Syndrome or cognitive impairment. A cerebrovascular disorders may include, but is not limited to stroke or vascular dementia.

In one aspect, there is provided a method for simulating or modeling a disorder associated with abnormal amyloid-beta (Aβ) protein uptake and clearance, comprising the method as defined herein.

In one aspect, there is provided a method for screening a compound for ability to treat a condition associated with abnormal Aβ protein uptake and clearance, comprising the steps of:

(A) exposing the brain-specific vascular SMCs prepared according to the method as defined herein to the hypoxic condition specified as defined herein to induce a disease phenotype associated with abnormal Aβ protein uptake and clearance,
(B) contacting the exposed brain-specific vascular SMCs with said compound,
(C) further exposing the brain-specific vascular SMCs that have been contacted with said compound to Aβ protein for a time sufficient to enable Aβ uptake and clearance, and
(D) measuring the level of Aβ uptake and clearance in said brain-specific vascular SMCs and comparing the level of Aβ uptake and clearance in said brain-specific vascular SMCs with the level of Aβ uptake and clearance in brain-specific vascular SMCs that have not been contacted with said compound,
wherein an increased level of Aβ uptake and clearance in said brain-specific vascular SMCs that have been contacted with said compound compared to brain-specific vascular SMCs that have not been contacted with said compound indicates that said compound is useful for treating said condition, and
wherein a decreased or unchanged level of Aβ uptake and clearance in said brain-specific vascular SMCs that have been contacted with said compound compared to brain-specific vascular SMCs that have not been contacted with said compound indicates that said compound is not useful for treating said condition. For example, such a “compound” can be any molecule or combination of more than one molecule that has the ability to treat a condition associated with abnormal Aβ protein uptake and clearance. The present invention contemplates screens for synthetic small molecule agents, chemical compounds, chemical combinations, and salts thereof as well as screens for natural products, such as plant extracts or materials obtained from fermentation broths. Other molecules that can be identified using the screens of the invention include proteins and peptide fragments, peptides, nucleic acids and oligonucleotides, carbohydrates, phospholipids and other lipid derivatives, steroids and steroid derivatives, prostaglandins and related arachadonic acid derivatives, etc.

The level of Aβ uptake and clearance measured in said brain-specific vascular SMCs in step (D) may be further or alternatively compared to the level of Aβ uptake and clearance in brain-specific vascular SMCs prepared according to the method as defined herein that have not been exposed to hypoxic condition specified herein, or that have been exposed to 21% oxygen (optionally at 37° C.), wherein the level of increase in Aβ uptake and clearance in said brain-specific vascular SMCs in step (D) relative to that of the brain-specific vascular SMCs that have not been exposed to hypoxic condition or that have been exposed to 21% oxygen provides an indication of the level of efficacy of said compound for treating said condition.

The level of Aβ uptake and clearance measured in said brain-specific vascular SMCs in step (D) may be further or alternatively compared to the level of Aβ uptake and clearance in brain-specific vascular SMCs prepared according to the method as defined herein, and exposed to the hypoxic condition specified herein and a reference compound,

wherein the level of increase in Aβ uptake and clearance in said brain-specific vascular SMCs in step (D) relative to that of the brain-specific vascular SMCs that have been exposed to the hypoxic condition specified as defined herein and a reference compound, provides an indication of the level of efficacy of said compound for treating said condition relative to said reference compound. In one example, a reference compound may include, but is not limited to a statin, an antibiotic and an angiotensin receptor blocker. A statin may be atorvastatin or simvastatin. An antibiotic may be rifampicin or minocycline. An angiotensin receptor blocker may be losartan.

The condition associated with abnormal Aβ protein uptake may include, but is not limited to aging, neurological disorders, or cerebrovascular disorders. Neurological disorders may include, but are not limited to cerebral amyloid angioplasty, Alzheimer's disease, Down's Syndrome or cognitive impairment. A cerebrovascular disorder may include, but is not limited to stroke or vascular dementia.

The contacting of the brain-specific vascular SMCs with said compound in step (B) may be carried out for a duration that can include, but is not limited to about 24 hr to about 1 week, about 24 hr, about 36 hr, about 48 hr, about 60 hr, about 72 hr, about 4 days, about 5 days, about 6 days, or about 7 days. The temperature may be at about 37° C. In one embodiment, the duration is about 24 hr to about 1 week.

The Aβ protein in step (C) to which the brain-specific vascular SMCs are exposed to may be labeled with a detectable label. A detectable label may include, but is not limited to a fluorescent label, a radioactive label or magnetic label. The term “labeled”, may refer to direct labeling of Aβ protein by coupling (i.e., physically linking) a detectable substance to the protein, as well as indirect labeling of the protein by reactivity with another reagent that is directly labeled. Examples of indirect labeling include detection of a primary antibody using a fluorescently labeled secondary antibody and end-labeling of the Aβ protein with biotin such that it can be detected with fluorescently labeled streptavidin.

The exposing in step (C) may be carried out for a duration that can include, but is not limited to about 30 min to about 12 hr, about 30 min, about 40 min, about 50 min, about 60 min, about 70 min, about 80 min, about 90 min, about 2 hr, about 2.5 hr, about 3 hr, about 4 hr, about 5 hr, about 6 hr, about 7 hr, about 8 hr, about 9 hr, about 10 hr, about 11 hr, or about 12 hr. The temperature may be at about 37° C., and wherein the Aβ protein to which the brain-specific vascular SMCs are exposed to is present in a concentration that can include, but is not limited to about 0.5 μg/ml to about 5 μg/ml, about 0.5 μg/ml, about 1 μg/ml, about 1.5 μg/ml, about 2 μg/ml, about 2.5 μg/ml, about 3 μg/ml, about 3.5 μg/ml, about 4 μg/ml, about 4.5 μg/ml or about 5 μg/ml. In one embodiment, the exposing in step (c) is to be carried out for a duration of about 60 min. In another embodiment, the Aβ protein to which the brain-specific vascular SMCs are exposed to is present in a concentration of about 2 μg/ml.

The level of Aβ uptake and clearance in said brain-specific vascular SMCs may be measured using a method that can include, but is not limited to fluorometric measurement, enzyme-linked immunosorbent assay (ELISA) or quantitative fluorescence microscopy. In one example, the fluorometric measurement is with flow cytometry.

In one aspect, there is provided a method for treating a patient in need of vascularized tissue replacement therapy, comprising administering to said patient the brain-specific vascular smooth muscle cells prepared according to the method as defined herein.

In one aspect, there is provided a method of analyzing blood vessel development using the brain-specific vascular smooth muscle cells prepared according to the method as defined herein.

In one aspect, there is provided a method of determining the suitability of a compound for treating a patient having a condition associated with abnormal Aβ uptake and clearance, the method comprising preparing brain-specific vascular smooth muscle cells according to the method as defined herein using a population of stem cells derived from said patient in step (a), subjecting said brain-specific vascular smooth muscle cells to the method as defined herein to induce a disease phenotype associated with abnormal Aβ uptake and clearance, and determining the suitability of said compound for treating said patient using the method as defined herein.

In one aspect, there is provided a composition comprising a BMP antagonist which is noggin present in a concentration of about 200 ng/ml, a FGF which is bFGF present in a concentration of about 12 ng/ml, an activin or nodal inhibitor which is SB431542 present in a concentration of about 10 μM.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−30 of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

The accompanying drawings illustrate a disclosed embodiment and serves to explain the principles of the disclosed embodiment. It is to be understood, however, that the drawings are designed for purposes of illustration only, and not as a definition of the limits of the invention.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

### EXAMPLES

Non-limiting examples of the invention, including the best mode, and a comparative example will be further described in greater detail by reference to specific Examples, which should not be construed as in any way limiting the scope of the invention.

### Example 1

### General Methods

### HPSC Maintenance

H9 HESC line (WiCell) was cultured in a chemically defined medium (CDM) as previously described (Brons et al., 2007). Human iPSCs were obtained by reprogramming human foreskin fibroblasts (BJs) into transgene-free iPSCs by the Sendai viral vector method. These BJ-iPSCs were grown on irradiated mouse feeders and cultured in DMEM/F12 medium, containing 20% Knockout serum replacement (Gibco) and 4 ng/ml FGF-2 (R&D Systems). Commercially available human iPSCs (KYOUDXR0109B, ATCC) were also used. These were derived from dermal fibroblasts obtained from a healthy adult donor. These fibroblasts were reprogrammed by the expression of OCT4, SOX2, KLF4, and MYC using retroviral transduction and subsequently cultured in the conditions as stated above.

### NCSMC Differentiation

For neural crest differentiation, hPSCs were grown in CDM+bFGF (12 ng/ml, R&D systems)+SB431542 (10 mM, Sigma)+noggin (200 ng/ml, R&D Systems) for 10-13 days, with change of medium every 2-3 days. After which, NGFR⁺/B3GAT1⁺ cells were isolated by fluorescence-activated cell sorting (FACS) and replated for SMC induction. Sorted cells were cultured in SMC differentiation medium CDM+PDGF-BB (10 ng/ml, PeproTech)+TGF-b1 (2 ng/ml, PeproTech) for another 12 days. Derived NCSMCs could be propagated further in culture using SMC medium (SC-1101, Sciencell). All experiments were done on NCSMCs between passages 1 and 9. On the other hand, the other SMC subtypes (NESMC, LMSMC, and PMSMC) were generated by a previously established protocol (Cheung et al., 2012).

### Source of BVSMCs

The human brain vascular smooth muscle cells (BVSMCs) were acquired from a commercial source, ScienCell Research Laboratories (cat no. #1100) and cultured using SMC medium (SC-1101, Sciencell). All experiments were done on BVSMCs between passages 1 and 9. The SMC medium contains essential and nonessential amino acids, vitamins, organic and inorganic compounds, hormones, growth factors, trace minerals and fetal bovine serum, smooth muscle growth supplements, and penicillin/streptomycin solution.

### Aβ Peptide Uptake Measurement by Flow Cytometry

SMC subtypes were incubated with 2 μg/ml of fluorescently labeled amyloid peptide (Aβ42-Hilyte Fluor 555 or Aβ40-Hilyte Fluor 488, Anaspec) at 37° C. for a 3 hr uptake unless otherwise stated. After Aβ uptake, cells were dissociated into single-cell suspension for flow cytometric analysis by Becton Dickinson FACSCalibur. Mean fluorescence brightness was calculated from each Aβ uptake sample in triplicates and normalized to the mean fluorescence brightness of no-uptake control samples. Relative comparisons of cell-associated Aβ in SMC subtypes were then plotted.

### High-Throughput Aβ Uptake Assay Development

NCSMCs and BVSMCs were routinely cultured at 21% oxygen and 1% oxygen to simulate normoxic and chronic hypoxic conditions, respectively. Twenty one percent O₂ SMCs were seeded at 30,000 cells/well onto 96-well plate as positive controls. One percent O₂ SMCs were seeded at the same density as negative controls and experimental samples for drug treatment on the same plate. The 96-well plate was incubated at 37° C. overnight at 21% O2. After cell attachment, 2 mg/ml of fluorescently labeled amyloid peptide (Aβ42-Hilyte Fluor 555 or Aβ40-Hilyte Fluor 488, Anaspec) was added to SMCs for a 1 hr uptake at 37° C. After Aβ uptake, cells were dissociated into single-cell suspension for high-throughput flow cytometry by LSRFortessa plate analyzer. Respective Z factors for NCSMC and BVSMC Aβ uptake assays were calculated based on their mean fluorescence brightness (normalized to no-uptake controls) and SDs of positive (21% O₂) and negative (1% O₂) controls. For the experimental samples with drug treatment, NCSMCs were incubated with reference compounds (i.e., atorvastatin, simvastatin, rifampicin, minocycline, losartan, and vehicle control) for 48 hr prior to Aβ uptake. Mean fluorescence brightness signals (normalized to no-uptake control) indicated the degree of amyloid uptake.

### Statistical Analysis

Results are presented as mean±SEM of three independent experiments unless otherwise stated. Statistical p values were calculated by Student's t test for two groups or by ANOVA for three or more groups. Significant differences are indicated as *p<0.05, **p<0.01, and **p<0.001 unless otherwise stated.

### Quantitative Real-Time Polymerase Chain Reaction (QRTPCR)

Total RNA was extracted with RNeasy Mini kit according to the manufacturer's instructions (QIAGEN). cDNA was prepared using Maxima First Strand cDNA Synthesis Kit (Fermentas). QRTPCR mixtures were prepared with SYBR Green PCR Master Mix (Applied Biosystems). QRTPCR reactions were performed in technical triplicates with the 7500 Fast Real-time PCR System (Applied Biosystems), using the Quantitation—comparative CT settings. Obtained values were normalised to GAPDH. Primer sequences are listed below.

### Genes

### Forward and Reverse Sequences

| | | SEQ |
|---|---|---|
| | Forward and reverse | ID |
| Genes | sequences | NO: |
| PAX3 | AGCTCGGCGGTGTTTTTATCA | 1 |
| | CTGCACAGGATCTTGGAGACG | 2 |
| SNAI1 | ACTGCAACAAGGAATACCTCAG | 3 |
| | GCACTGGTACTTCTTGACATCTG | 4 |
| SNAI2 | TGTGACAAGGAATATGTGAGCC | 5 |
| | TGAGCCCTCAGATTTGACCTG | 6 |
| NGFR | CCTACGGCTACTACCAGGATG | 7 |
| | CACACGGTGTTCTGCTTGT | 8 |
| B3GAT1 | CTCCTTCGAGAACTTGTCACC | 9 |
| | GGGTCAGTGAAGCCCTTCTT | 10 |
| TFAP2A | ACTTTGGGTACGTGTGCGAAA | 11 |
| | CGGAATGTTGTCGGTTGAGAAAT | 12 |
| SOX10 | CCTCACAGATCGCCTACACC | 13 |
| | CATATAGGAGAAGGCCGAGTAGA | 14 |
| LRP1 | GAGCTGAACCACGCCTTTG | 15 |
| | GGTAGACACTGCCACTCCGATAC | 16 |
| LDLR | GGACCAACGAATGCTTGGACA | 17 |
| | CTGGCACTTGTAGCCACCC | 18 |
| MME | AGAAGAAACAGCGATGGACTCC | 19 |
| | CATAGAGTGCGATCATTGTCACA | 20 |
| PLAT | ATGGATGCAATGAAGAGAGGGC | 21 |
| | CTGGGCGAAACGAAGACTG | 22 |

### Immunocytofluorescence and Flow Cytometry

For immunofluorescence, adherent cells were fixed and permabilised. After blocking with 3% BSA, primary antibody was incubated at 4° C. overnight. Secondary goat anti-mouse Alexa Fluor 568 and goat anti-rabbit Alexa Fluor 488 antibodies (Invitrogen Molecular Probes) were used. Finally, nuclei were stained with DAPI. Images were acquired by live cell imaging microscope (Zeiss Axiovert 200M). For flow cytometry, harvested cells were fixed using the Cytofix/Cytoperm Fixation/Permeabilization kit (BD Biosceinces) and stained according to the kit manual. Mouse IgG isotype controls (BD PharMingen, 555749, R&D Systems, IC002C) and non-expressing cell controls (NE, NC and HUVECs) were used for certain experiments. Primary antibodies used for this study are listed as followed.

| | | Working | Source |
|---|---|---|---|
| Proteins | Application | dilution | (cat. number) |
| NGFR | Flow cytometry | 1:20 | Biolegend |
| | | | (345109) |
| B3GAT1 | Flow cytometry | 1:20 | Biolegend |
| | | | (245107) |
| TAGLN | Immunostaining | 1:600 | Abcam |
| | | | (ab14106) |
| ACTA2 | Flow cytometry | 1:500 | Sigma |
| | | | (F3777) |
| TUBB3 | Immunostaining | 1:100 | Abcam |
| | | | ab18207 |
| MAP2 | Immunostaining | 1:500 | Abcam |
| | | | ab11267 |
| HIF1A | Immunostaining | 1:500 | Sigma |
| | | | (SAB2101039-50UG) |
| LRP1 | Flow cytometry | 1:20 | My BioSource |
| | | | MBS135013 |
| LDLR | Flow cytometry | 5 μL/106 cells | R&D Systems |
| | | | (FAB2148G) |

### Microarray Analysis

The Illumina HumanHT-12 v4 BeadArray was used to obtain global gene expression of hESC and SMC samples. Acquired data was preprocessed, log 2-transformed and quantile normalised using the beadarray package in Bioconductor. Unsupervised hierarchical clustering was performed by using complete linkage clustering with a Spearman's correlation coefficient as similarity metric. Gene ontology analysis was performed using the functional annotation clustering from DAVID bioinformatics resources.

### Hypoxia Treatment

SMCs were cultured in a 37° C. incubator at 1% oxygen for at least 2 weeks to induce chronic hypoxia. These SMCs were routinely fed with SMC medium (SC-1101, Sciencell) which have been conditioned to 1% oxygen for at least 48 hours.

### SMC Contraction Study

SMCs were preloaded with the calcium-sensitive fluorophore, Fluo-4 AM (2.5 μM; Molecular Probes) at 37° C. for an hour. Contraction was induced by treating the cells with angiotensin II (0.5 μM, Sigma). Contraction pictures and intracellular calcium flux videos of SMCs were acquired by a time-lapse microscope live cell imaging microscope (Zeiss Axiovert 200M) during 10 minutes of angiotensin II treatment. Change of cell surface area was assessed by ImageJ software.

### Neuronal Differentiation

We carried out neuronal differentiation on our NGFR+/B3GAT1+ neural crest population according to an established protocol (Hu and Zhang, 2009). Briefly, the neural crest cells were treated with retinoic acid (100 nM) for 5 days, followed by both retinoic acid and sonic hedgehog for another 2 weeks. After which, the neuronal progenitors that emerged were re-plated onto laminin substrate for differentiation into post-mitotic motor neurons. Cells were cultured in the neurobasal medium supplemented with brain-derived neurotrophic factor, glial-derived neurotrophic factor, insulin-like growth factor-1, and cAMP for at least another week. Subsequently, we performed immunostaining for neuronal markers, TUBB3 and MAP2.

### Gene Silencing of LRP1

Transient knockdown of LRP1 was carried out with ON-TARGETplus SMARTpool siRNA by DharmaFECT transfection reagent (Thermo Scientific Dharmacon). 25 nM of siRNA was used for transfection according to the manufacturer's instructions. Messenger RNA and protein analyses were performed at 48 hours and 72 hours post-transfection respectively.

### Intracellular Aβ Measurement by ELISA

NCSMC were incubated with 5 μg/mL of human recombinant Aβ42 or Aβ40 (Anaspec) at 37° C. for a 3-hour uptake. The cells were then washed twice with ice-cold PBS and total proteins from cell lysates were collected using RIPA buffer (Thermo Scientific) at 0 hour, 2 days and 5 days after Aβ uptake. Protein quantification was performed by the Quant-iT Protein Assay Kit (Invitrogen). ELISA was performed using 30-100 μg of protein per sample on the human Aβ40 and Aβ42 ELISA kits (Invitrogen), according to manufacturer's instruction. The optical density was recorded at 450 nm using a microplate reader (Tecan Infinite M200) to determine the concentrations of intracellular Aβ40 and Aβ42.

### Imaging of Lysosomal Internalisation of Aβ

NCSMC were first incubated with 1 μM of LysoTracker® Blue DND-22 (Life Technologies) for 2 hours. Cell culture was washed and replaced by fresh medium with 2 μg/mL of fluorescently labelled amyloid peptide (Aβ42-Hilyte Fluor 555 or Aβ40-Hilyte Fluor 488, Anaspec). Cells were incubated at 37° C. for another 3-hour uptake and then imaged using a fluorescence microscope (Zeiss Axiovert 200M).

### Annexin V Conjugates for Apoptosis Detection

Apoptosis analysis was performed using annexin V conjugates (Invitrogen). After NSMC were treated with human recombinant Aβ peptides, cells were collected and diluted in annexin-binding buffer (10 mM HEPES, 140 mM NaCl, and 2.5 mM CaCl2, pH 7.4) to a density of 1 million cells/ml. 5 μL of the annexin V conjugate was then added to each 100 μL of cell suspension. Cells were incubated at room temperature for 15 minutes. After the incubation period, 400 μL of annexin-binding buffer was added to mix gently. Samples were kept on ice until flow cytometric analysis.

### Drug Treatment

NCSMC were treated with the following reference compounds for 48 hours at 37° C. prior to Aβ uptake. The working concentration used were atorvastatin (0.5 μM, Sigma), simvastatin (0.5 μM, Sigma), rifampicin (5 μM, Sigma), minocycline (5 μM, Selleck Chemicals), losartan (5 μM, Sigma). Vehicle control was 0.1% DMSO in SMC medium (SC-1101, Sciencell).

### Example 2

### Derivation of Neural Crest-Specific Vascular SMCs

The majority of studies to date have focused on the generation of generic SMCs, which may confound potential lineage-specific differences. The inventors and others have attempted the derivation of origin-specific SMCs from hPSCs (Cheung et al., 2012). For the purpose of modeling cerebral SMCs, different chemical cocktails were tested based on factors known to promote neural crest differentiation in hPSCs (FIG. 1A). Because activation of canonical Wnt signaling had been previously shown to induce neural crest formation in hPSCs, an inhibitor of glycogen synthase kinase 3, CHIR99021, was tested. Bone morphogenetic protein (BMP) antagonist noggin was included to counteract mesodermal induction due to endogenous BMP signaling in hPSCs. A combination of basic fibroblast growth factor(bFGF, 12 ng/ml), activin/nodal inhibitor SB431542 (10 mM), noggin (200 ng/ml) resulted in peaked expressions of neural crest markers between days 10 and 16 of hPSC (hESC H9) differentiation(FIG. 2A). The development of neural crest surface markers was monitored (FIG. 1B) and it was found that both FSb(bFGF+SB431542) and FSbN (bFGF+SB431542+noggin) conditions yielded substantial amount (>65%) of double NGFR and B3GAT1 expressing cells on day 10. Isolated NGFR+/B3GAT1+ cells from FSbN treatment confirmed greater expression levels of neural crest genes than those from FSb treatment(FIG. 2B). Hence, these NGFR+/B3GAT1+ cells were purified, following FSbN treatment, to perform subsequent differentiation into vascular SMCs.

A previous SMC differentiation protocol(Cheung et al., 2012) was adopted to differentiate this intermediate neural crest population using platelet-derived growth factor BB(PDGF-BB, 10 ng/ml) and transforming growth factor-beta 1(TGF-b1, 2 ng/ml) for another 12 days. The resultant neural crest-derived SMCs (NCSMCs) were then characterized in comparison to neuroectoderm-derived SMCs (NCSMCs) (Cheung et al., 2012) and positive control, human brain vascular SMCs (BVSMCs). The source of BVSMCs used in this work has been previously employed in several studies as a model of human cerebrovascular SMCs. A panel of SMC-related genes was upregulated in all SMC subtypes derived from both hESCs (H9) and iPSCs (BJ-iPSCs) as compared to hPSCs (FIG. 2C). NCSMCs demonstrated comparable levels of TAGLN (FIG. 2D), an actin crosslinking protein found in smooth muscle, and contractile protein ACTA2 (FIG. 1E), as NESMCs and BVSMCs. Furthermore, NCSMCs exhibited contractile ability with reduction of cell surface area in response to a vasoconstrictor, angiotensin II (FIG. 2F). Waves of calcium influx were detected by fluo-4 imaging during contraction (FIG. 1C). These results verified that the NCSMCs were functional. To determine whether our NGFR+/B3GAT1+ neural crest population was indeed multipotent, neuronal differentiation was carried out (Hu and Zhang, 2009). The neural crest cells were found to give rise to network structures stained positive for neuronal markers TUBB3 and MAP2 (Figure S1D).

### Example 3

### NCSMCs Demonstrate Cerebrovascular-Relevant Amyloid-β Uptake Ability

To model after cerebrovascular SMCs with high fidelity, we investigated whether NCSMCs shared the closest molecular signatures and functional characteristics with BVSMCs than the SMC subtypes originating from other embryonic origins (e.g., neuroectoderm, lateral plate mesoderm, and paraxial mesoderm) (Cheung et al., 2012). Unless otherwise stated, most experiments were performed using SMC subtypes were derived hESC (H9). Gene expression analysis showed that NCSMCs derived from either hESCs (H9) or iPSCs (BJ-iPSCs) clustered more closely with BVSMCs than NESMCs in the gene ontology (GO) category of brain development (FIG. 3A; genes listed in Table 1). Functional annotation of the commonly enriched genes in all SMC subtypes indicated GO categories such as blood vessel development and extracellular matrix organization as expected (FIG. 4A). Common genes specific only to BVSMCs and NESMCs demonstrated functions pertaining to blood vessel development, but there were also irrelevant GO categories like skeletal system and bone development. On the other hand, NESMCs seemed to share more specialized functions with BVSMCs in integrin-mediated pathway (adhesion), epithelial cell differentiation (necessary for neural crest specialization), response to calcium (contractile ability), and smooth muscle development. Hence, it was postulated that BVSMCs could resemble more closely to NESMCs than NESMCs. Because oxygen deprivation could be an initiating event in the pathogenesis of CAA, various SMC subtypes were cultured at 1% oxygen for at least 2 weeks to induce chronic hypoxia. All the SMC subtypes responded with an upregulation of hypoxiainducible factor-1a (FIG. 4B). Internalization of Aβ by lipoprotein receptors on SMCs was reported to be compromised by hypoxic conditions. Low-density lipoprotein receptor (LDLR) was previously found to mediate Aβ uptake and cell death of cerebral perivascular cells. Cerebrovascular cells and neurons also express different Aβ-degrading enzymes including neprilysin (MME), tissue plasminogen activator (PLAT), and matrix metalloproteinases. MME is downregulated in both hypoxia and Alzheimer's disease patients manifesting CAA. By gene expression quantification, it was found that LRP1, LDLR, MME, and PLAT were significantly downregulated in a majority of the SMC subtypes, with the greatest reduction observed in NESMCs and NESMCs compared to their counterparts that were cultured at 21% oxygen (FIG. 3B). Protein analysis showed decreased percentage of LRP1-expressing cells in the SMC subtypes at 1% oxygen (FIG. 3C). LRP1 is abundantly expressed in the cerebral vasculatures, in particular, the SMCs (Lillis et al., 2008). It was found that more than 50% of NCSMCs expressed LRP1, constituting to the highest percentage of LRP1+ cells among the SMC subtypes (FIG. 3C, right panel). Taking into consideration the difference in abundance of LRP1 protein per cell, the median fluorescent intensity of LRP1-FITC in each of the SMC populations also decreased significantly at 1% oxygen (FIG. 4C).

Aβ40 is the chief form of vascular amyloid component in CAA, whereas Aβ42 may be responsible for early-stage damage. Aβ deposits in parenchyma and blood vessels seem to be originated from neuronal-derived Aβ as animal studies previously indicated that local production of Aβ by cerebrovascular cells was not essential to drive CAA pathology. To examine hPSC-derived SMCs′ability to uptake exogenous Aβ, BVSMCs were first treated with fluorescently labeled Aβ40 and Aβ42 peptides (2 mg/ml). Aβ internalization via LRP1 requires the presence of serum lipoprotein. At different time points after the onset of Aβ uptake in serum-containing media, the mean fluorescence brightness was measured by flow cytometry to determine the amount of cell-associated Aβ in BVSMCs (FIG. 4D). The rate of Aβ uptake slowed down after 3 hr, and so 3 hr Aβ uptake was carried out for subsequent experiments. To investigate the effect of hypoxia on uptake of exogenous Aβ, all the SMC subtypes were treated with fluorescently labeled Aβ40 and Aβ42 peptides. In compliance with an abundance of LRP1 expression found in cerebral vasculatures (Lillis et al., 2008), and the highest LRP1 expression showed in FIG. 3C, NCSMCs demonstrated greatest uptake of Aβ40 and Aβ42 at 21% oxygen (FIG. 4E). After the cells were cultured at 1% oxygen, hypoxia attenuated the amount of cell-associated Aβ, especially for NCSMCs (Aβ40, p=0.000324; Aβ42, p=0.000723) and NCSMCs (Aβ40, p=0.000229; Aβ42, p=0.000556) (FIG. 3D). On the other hand, the uptake of Aβ40 was not compromised in LMSMCs or PMSMCs under 1% oxygen, suggesting that such mesodermal derivatives may not mediate Aβ uptake primarily through LRP1-dependent pathway. Differences in Aβ metabolism among the SMC subtypes seemed to have already been influenced early by the differences in spatiotemporal development of neural crest and mesoderm. Non-neural crest SMC subtypes may not fully recapitulate the biology of cerebrovascular-specific attributes.

It was further tested if in vitro Aβ metabolism could be assayed under more physiological conditions. Uptake of Aβ in NCSMCs increased in a concentration-dependent manner (FIG. 4F). 300 ng/ml was chosen as a near-physiological concentration, which yielded a relatively well-detectable readout to proceed with Aβ uptake under 1% oxygen. Consistent with previous experiments, hypoxia attenuated the amount of cell-associated Aβ40 and Aβ42 in BVSMCs (Aβ40, p=0.00105; Aβ42, p=0.00304) and NCSMCs (Aβ40, p=0.00015; Aβ42, p=0.00290) (FIG. 4G). However, no significant change was observed in NESMCs, suggesting that hypoxia-induced alteration to Aβ uptake at physiological range may not be reproducible in NESMCs. Hence, NCSMC was the most appropriate SMC subtype for reproducing the pathophysiological features of cerebral vasculatures in Aβ metabolism.

### Example 4

### NCSMCs Recapitulate LRP1-Mediated Amyloid-β Clearance

SMC-specific knockout of Lrp1 in an amyloid mouse model is known to accelerate brain Aβ accumulation and CAA. LRP1 suppression in brain primary SMCs significantly diminished their ability to uptake and degrade exogenous Aβ. To study whether Aβ processing in NCSMCs was regulated by LRP1, gene silencing of LRP1 was performed using small interfering RNA (siRNA). At least 3-fold reduction of LRP gene (p=0.0120) (FIG. 5A) and protein levels (p=0.00144) (FIG. 6A) was achieved in comparison to the scrambled siRNA controls. LRP1 knockdown resulted in compromised uptake of both Aβ40 (p=0.0000558) and Aβ42 (p=0.000101) in NCSMCs (FIG. 6B). Intracellular lysosomal degradation of Aβ following uptake is also known to depend on the presence of LRP1. The use of ELISAs was used to study Aβ clearance after cellular uptake. LRP1 siRNA- and scrambled siRNA-treated NCSMCs were first incubated with mg/ml of human recombinant Aβ for 3 hr in serum-containing medium. Total cell lysates were then collected at days 0, 2, and 5 after uptake for analysis. NCSMCs had low endogenous Aβ before uptake (FIG. 6C). After uptake, scrambled control NCSMCs possessed significantly higher amounts of Aβ40 (p=0.00103) and Aβ42 (p=0.0224) than the LRP1 siRNA-treated NCSMCs. Intracellular Aβ decreased over time with LRP1 siRNA-treated NCSMCs showing slower rate of Aβ42 clearance than scrambled siRNA-treated NCSMCs (FIG. 6D). By day 5, degradation of both Aβ40 (p=0.0495) and Aβ42 (p=0.0432) was significantly compromised by LRP1 silencing in NCSMCs. In the LRP1 siRNA-treated NCSMCs, different rate of degradation was observed with Aβ40 being cleared more efficiently than Aβ42. This could suggest that the presence of Aβ-degrading proteases could contribute to the differential degradation kinetics of distinct forms of Aβ. Clearance of Aβ peptides was also affected in the positive control BVSMCs as evident in the significantly higher amounts of remnant Aβ40 (p=0.00336) and Aβ42 (p=0.000291) in the LRP1 siRNA-treated BVSMCs after 2 days of degradation (FIG. 5B). Other SMC subtypes appeared to have inconsistent extent of Aβ clearance in response to LRP1 silencing, indicating that SMCs of other embryonic origins might not fully recapitulate lipoprotein receptor-mediated Aβ metabolism.

To investigate whether LRP1 played a role in lysosomal degradation of Aβ in our NCSMCs, a lysotracker dye was used to image for any colocalization of fluorescently labeled Aβ with lysosomes. Live cells were monitored at days 0, 3, and 7 after 3 hr of Aβ uptake. The results supported the above finding that LRP1 siRNA treated NCSMCs had compromised Aβ clearance as traces of incorporated Aβ in lysosomes were still found at days 3 and 7 but not for the control NCSMCs (FIG. 6E). Like previously described in human cerebrovascular SMCs, it was confirmed that LRP1 functioned in our NCSMCs to facilitate intracellular Aβ degradation. The involvement of other lipoprotein receptors and Aβ-degrading proteases in Aβ clearance can be further explored using our NCSMC system. Amyloid pathology in the brain is often accompanied by SMC atrophy (Blaise et al., 2012). The effect of recombinant Aβ on cell death of NCSMCs was also studied. Hypoxic condition exacerbated apoptosis of NCSMCs as evident by annexin V staining (FIG. 5C). Higher concentrations of recombinant Aβ only resulted in minor increase in the proportion of apoptotic cells at 1% oxygen. The limitation of modeling Aβ induced SMC death in vitro was recognized, as substantial SMC loss in the leptomeningeal artery of patients was a consequence of chronic exposure to Aβ deposits. Hence, for the aim of improving vascular health, NCSMCs could be used to develop a phenotypic assay of Aβ uptake.

### Example 5

### High-Throughput Assay of Amyloid-3 Uptake for Screening of Reference Compounds

To attain the eventual goal of cell-based phenotypic screening, a panel of reference pharmaceutical compounds was selected for testing. Patients on statin prescriptions for lowering circulating cholesterol to prevent cardiovascular diseases were previously reported to have lower incidence of neurodegenerative disorders and improved cognitive performance, probably due to pleiotropic vascular protective effects of drugs. Statins prevent late-onset Alzheimer's disease by stimulating LRP1 expression on brain vascular cells. Rifampicin, an antibiotic, is known to enhance Aβ clearance by inducing LRP1 expression at the blood-brain barrier. Another antibiotic, minocycline (Choi et al., 2007), and angiotensin receptor blockers are able to attenuate the progression of dementia, but there is not yet direct evidence that they target the Aβ clearance machinery. The reference compounds were tested on NCSMCs cultured at 1% oxygen and it was found that statins and rifampicin upregulated the gene and protein expressions of lipoprotein receptors (FIGS. 7A and 7B). These drugs were further evaluated for their potential benefits on Aβ uptake in NCSMCs.

To develop high-throughput screening capability on the NCSMCs, a sensitive and disease-relevant readout had to be chosen, in this case, the uptake of labeled Aβ peptides. The NCSMC and BVSMC cultures were scaled down to 96-well format. A plating density of 30,000 cells/well yielded good post-thawing survival and reproducibility for assessment of Aβ uptake. SMCs, which were routinely grown at 21% oxygen, represented the positive control, whereas SMCs conditioned to chronic hypoxia at 1% oxygen were used as the negative control and experimental samples. Cells were incubated with labeled Aβ40 and Aβ42 (2 mg/ml) for an hour and subsequently evaluated by high-throughput flow cytometry. Aβ uptake in NCSMCs derived from both hESC (H9) and iPSC (KYOUDXR0109B) resulted in Z-factors of more than 0.5, indicative of a statistically robust assay (FIG. 7C). On the other hand, BVSMC produced a Z-factor between 0 and 0.5, which was considered only a marginal assay. To prove the feasibility of drug screening, NCSMCs derived from hESC (H9) were treated with reference compounds and vehicle control for 48 hr, followed by uptake of Aβ for another hour. Among these reference compounds, only statins and rifampicin were previously reported to exert direct effects on upregulating LRP1 and LDLR expression. Our findings revealed that atorvastatin (p=0.0492) and rifampicin (p=0.0174) significantly increased Aβ40 and Aβ42 uptake, respectively, in NCSMCs over the vehicle controls (FIG. 7D). This could be due to increased expression of the lipoprotein receptors although the effects of such compounds on Aβ uptake may occur through other mechanisms as well. Minocycline and losartan may not have any apparent effect and could be further investigated by a dose-response experiment. Drug combinations appeared to be counter-effective because there were no significant beneficial effects over the control. Likewise, NCSMCs derived from human iPSC (KYOUDXR0109B) had identified rifampicin, which could improve Aβ uptake (FIG. 5D), supporting the robustness of NCSMC model for phenotypic screening. Taken together, a cell-based platform that is amenable for high-throughput screening of novel compounds and for unraveling beneficial effects of vascular protective compounds has been established.

All referenced publications and patent documents are incorporated by reference herein, as though individually incorporated by reference.

### REFERENCES

Brons, I. G., Smithers, L. E., Trotter, M. W., Rugg-Gunn, P., Sun, B., Chuva de Sousa Lopes, S. M., Howlett, S. K., Clarkson, A., Ahrlund-Richter, L., Pedersen, R. A., and Vallier, L. (2007). Derivation of pluripotent epiblast stem cells from mammalian embryos. Nature 448, 191-195.
Cheung, C., Bernardo, A. S., Trotter, M. W., Pedersen, R. A., and Sinha, S. (2012). Generation of human vascular smooth muscle subtypes provides insight into embryological origin-dependent disease susceptibility. Nat. Biotechnol. 30, 165-173.
Hu, B. Y., and Zhang, S. C. (2009). Differentiation of spinal motor neurons from pluripotent human stem cells. Nat. Protoc. 4, 1295-1304.

What is claimed is:

1. A method for preparing brain-specific vascular smooth muscle cells, the method comprising the step of:
(a) contacting a population of stem cells with a composition comprising a bone morphogenetic protein (BMP) antagonist, a fibroblast growth factor (FGF) and an activin or nodal inhibitor to produce a population of neural crest cells.

2. The method according to claim 1, wherein the composition in step (a) does not comprise any one of the following which is selected from the group consisting glycogen synthase kinase (GSK) inhibitor, a Wnt protein, and a composition that is serum-free.

3. The method according to claim 1, further comprising the step of:
(b) isolating a population of CD57+ and/or CD271+ neural crest progenitor cells from the population of neural crest cells produced in step (a).

4. The method according to claim 1, further comprising the step of:
(c) contacting the population of neural crest progenitor cells with a composition comprising platelet-derived growth factor (PDGF) and transforming growth factor (TGF) to produce said brain-specific vascular smooth muscle cells.

5. The method according to claim 1, wherein said population of stem cells in step (a) are human pluripotent stem cells.

6. The method according to claim 5, wherein said human pluripotent stem cells are selected from the group consisting of human embryonic stem cells (hESCs) and induced pluripotent stem cells (IPSCs).

7. The method according to claim 6, wherein said human pluripotent stem cells are derived from stem cell lines and/or via stem cell preparation methods that do not involve destruction of human embryos.

8. The method according to claim 1, wherein the contacting of the population of stem cells with the composition in step (a) is carried out in a monolayer of said population of stem cells.

9. The method according to claim 1, wherein the composition in step (a) comprises:
(i) a BMP antagonist selected from the group consisting of noggin, an inhibitor of the transcriptional activity of the BMP type I receptors ALK2 and/or ALK3, chordin, and LDN193189; and/or
(ii) a FGF which is selected from the group consisting of a basic fibroblast growth factor (bFGF), FGF-17, FGF-5, FGF-16, FGF-6, FGF-20, FGF-12, FGF-4, FGF-10, FGF-21, FGF-8a, FGF-23, FGF-9, FGF-19, FGF-22, and FGF-3; and/or
(iii) an activin or nodal inhibitor selected from the group consisting of SB431542, LY2157299, SB525334, SB505124 and LY2109761.

10. The method according to claim 1, wherein the composition in step (a) comprises:
(i) a BMP antagonist present in a concentration selected from the group consisting of about 100 ng/ml to about 500 mg/ml, about 100 ng/ml, about 200 ng/ml, about 300 ng/ml, about 400 ng/ml, and about 500 ng/ml; and/or
(ii) a FGF present in a concentration selected from the group consisting of about 5 ng/ml to about 20 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml and about 20 ng/ml; and/or
(iii) an activin or nodal inhibitor present in a concentration selected from the group consisting of about 5 μM to about 20 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, and about 20 μM.

11. The method according to claim 1, wherein the composition in step (a) comprises:
(i) a BMP antagonist which is noggin present in a concentration of about 200 ng/ml; and/or
(ii) a FGF which is bFGF present in a concentration of about 12 ng/ml; and/or
(iii) an activin or nodal inhibitor which is SB431542 present in a concentration of about 10 μM.

12. The method according to claim 1, wherein the contacting in step (a) is for a duration selected from the group consisting of about 8 days to about 18 days, about 10 days to about 16 days, about 8 days, about 9 days, about 10 days, about 11 days, about 12 days, about 13 days, about 14 days, about 15 days, about 16 days, about 17 days and about 18 days.

13. The method according to claim 3, wherein the population of CD57+ and/or CD271+ neural crest progenitor cells are isolated in step (b) using Fluorescence Assisted Cell Sorting (FACS) or magnetic assisted cell sorting (MACs).

14. The method according claim 4, wherein the composition in step (c) comprises:
(i) a PDGF which is platelet-derived growth factor BB (PDGF-BB); and/or
(ii) a TGF which is transforming growth factor-beta 1 (TGF-β1).

15. The method according claim 4, wherein the composition in step (c) comprises:
(i) a PDGF present in a concentration selected from the group consisting of about 5 ng/ml to about 20 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, and about 20 ng/ml; and/or
(ii) a TGF present in a concentration selected from the group consisting of about 1 ng/ml to about 5 ng/ml, about 1 ng/ml, about 1.5 ng/ml, about 1.6 ng/ml, about 1.7 ng/ml, about 1.8 ng/ml, about 1.9 ng/ml, about 2 ng/ml, about 2.1 ng/ml, about 2.2 ng/ml, about 2.3 ng/ml, about 2.4 ng/ml, about 2.5 ng/ml, about 3 ng/ml, about 4 ng/ml and about 5 ng/ml.

16. The method according to claim 4, wherein the contacting in step (c) is for a duration selected from the group consisting of about 9 days to about 20 days, about 10 days to about 16 days, about 10 days to about 14 days, about 9 days, about 10 days, about 11 days, about 12 days, about 13 days, about 14 days, about 15 days, about 16 days, about 17 days, about 18 days, about 19 days and about 20 days.

17. A method for inducing a disease phenotype associated with abnormal amyloid-beta (Aβ) protein uptake and clearance, the method comprising the step of exposing the brain-specific vascular SMCs prepared according to the method of claim 1 to hypoxic condition for a length of time sufficient to induce said disease phenotype.

18. The method according to claim 17, wherein the hypoxic condition comprises a condition selected from the group consisting of less than about 5% of oxygen, less than about 4% of oxygen, less than about 3% of oxygen, less than about 2% of oxygen, and less than about 1% of oxygen.

19. The method according to claim 17, where the length of time sufficient to induce said disease phenotype is selected from the group consisting of at least about 48 hr, at least about 3 days, at least about 4 days, at least about 5 days, at least about 6 days, at least about 1 week, at least about 2 weeks, at least about 3 weeks, at least about 4 weeks, at least about 2 months, at least about 3 months, at least about 4 months, at least about 5 months, about 2 weeks or more, about 3 weeks or more, about 4 weeks or more, about 2 months or more, about 3 months or more, about 4 months or more, and about 5 months or more.

20. The method according to claim 17, wherein the Aβ protein is selected from the group consisting of Aβ40 and Aβ42.

21. The method according to claim 17, wherein said disease phenotype is selected from the group consisting of aging, neurological disorders, and cerebrovascular disorders.

22. A method for simulating or modeling a disorder associated with abnormal amyloid-beta (Aβ) protein uptake and clearance, comprising the method according to claim 17.

23. A composition comprising a BMP antagonist which is noggin present in a concentration of about 200 ng/ml, a FGF which is bFGF present in a concentration of about 12 ng/ml, an activin or nodal inhibitor which is SB431542 present in a concentration of about 10 μM.

24. The composition according to claim 23, wherein the composition does not comprise any one of the following which is selected from the group consisting of a glycogen synthase kinase (GSK) inhibitor, a Wnt protein, and a serum-free composition.
